(19) United States
(12) Patent Application Publication
(10) Pub. No.: US 2022/0048987 A1
(43) Pub. Date: Feb. 17, 2022

(54) METHOD OF TREATMENT TO PREVENT OR REVERSE AGE-ASSOCIATED INFLAMMATION, COGNITIVE DECLINE, AND NEURODEGENERATION

(71) Applicant: The Board of Trustees of the Leland Stanford Junior University, Stanford, CA (US)

(72) Inventors: Katrin Andreasson, Stanford, CA (US); Paras Minhas, Stanford, CA (US)

(21) Appl. No.: 17/401,879

(22) Filed: Aug. 13, 2021

Related U.S. Application Data
(60) Provisional application No. 63/065,245, filed on Aug. 13, 2020.

(51) Int. Cl. C07K 16/26; A61P 25/00

(52) U.S. Cl. CPC C07K 16/26; A61K 2039/505; A61P 25/00

(57) ABSTRACT

In aging mice, myeloid cell bioenergetics are suppressed in response to increased signaling by the lipid messenger prostaglandin E2 (PGE2), a major modulator of inflammation. In aging macrophages and microglia, PGE2 signaling through its EP2 receptor promotes the sequestration of glucose into glycogen, reducing glucose flux and mitochondrial respiration. Inhibition of myeloid EP2 signaling restores youthful energy metabolism in peripheral macrophages and microglia, rejuvenates systemic and brain inflammatory states, and prevents loss of hippocampal synaptic plasticity and spatial memory. Blockade of peripheral myeloid EP2 signaling is sufficient to restore cognition in aged mice.

### CROSS-REFERENCE TO RELATED APPLICATION

This application claims the benefit of U.S. Provisional Application No. 63/065,245 filed on Aug. 13, 2020, the entire disclosure of which is incorporated herein by reference.

### STATEMENT REGARDING FEDERALLY SPONSORED RESEARCH OR DEVELOPMENT

This invention was made with Government support under contracts AG048232, AG058047, and NS087639 awarded by the National Institutes of Health. The Government has certain rights in the invention.

### TECHNICAL FIELD

The present disclosure is generally related to methods of reducing cognitive decline by small molecule administration.

### SEQUENCE LISTING

This application contains a sequence listing filed in electronic form as an ASCII.txt file entitled “2219071385_5 T25” created on Aug. 13, 2021. The content of the sequence listing is incorporated herein in its entirety.

### BACKGROUND

Aging is associated with a progressive increase in systemic inflammation that adversely affects brain function and increases susceptibility to neurodegenerative diseases like Alzheimer's disease. The consensus thus far for treating brain dysfunction, degeneration or disease has been that a brain-penetrant small molecule or other agent is needed, and that penetration of the blood brain barrier would be needed for treatment. Non-toxic treatments not requiring penetration of the blood brain barrier are sought after. These needs and other needs are satisfied by the present disclosure.

### SUMMARY

One aspect of the disclosure, therefore, encompasses embodiments of a method for reducing inflammation in a subject, wherein the inflammation is associated with neurological or cognitive decline in the subject, comprising inhibiting an EP2 (Prostaglandin E₂ receptor 2)-generated signal in the subject by contacting EP2 with an EP2 antagonist.

In some embodiments of this aspect of the disclosure, inhibiting the EP2 signal comprises administering to the subject a composition comprising a brain-penetrant EP2 antagonist, a peripheral EP2 antagonist, or both.

In some embodiments of this aspect of the disclosure, the EP2 antagonist is a small molecule antagonist.

In some embodiments of this aspect of the disclosure, the EP2 is in aged human monocyte-derived macrophages.

In some embodiments of this aspect of the disclosure, the brain-penetrant EP2 antagonist is compound 52.

In some embodiments of this aspect of the disclosure, the peripheral EP2 antagonist is PF04418948.

Another aspect of the disclosure encompasses embodiments of a method for reducing cognitive decline in a subject, comprising inhibiting an EP2-generated signal by administering a composition comprising an EP2 antagonist to a subject in need thereof.

In some embodiments of this aspect of the disclosure, the EP2-generated signal is a myeloid EP2-generated signal.

In some embodiments of this aspect of the disclosure, the myeloid EP2-generated signal is inhibited in aged mammalian monocyte-derived macrophages.

In some embodiments of this aspect of the disclosure, the composition is administered to the mammal peripherally.

In some embodiments of this aspect of the disclosure, administering is oral or intravenous.

Yet another aspect of the disclosure encompasses embodiments of a pharmaceutical composition comprising an EP2 antagonist and a pharmaceutically acceptable carrier, wherein the pharmaceutical composition is formulated to deliver an effective dose of the antagonist to the mammal that inhibits an EP2-generated signal in the cells thereof.

In some embodiments of this aspect of the disclosure, the therapeutically effective amount is effective to reduce brain and/or peripheral myeloid EP2-generated signaling.

### BRIEF DESCRIPTION OF THE DRAWINGS

FIGS. 1A-1J illustrate that PGE2 regulates myeloid cell metabolism and in vivo inflammation through the EP2 receptor in aged mice.

FIG. 1A PGE2 levels were measured in cell lysates and in medium from young (<35 years) and aged (>65 years) human monocyte-derived macrophages (human MDMs) cultured for 20 hours (n=5 biologically independent samples; **** P<0.0001. See FIGS. 5A-5B.

FIG. 1B shows representative traces of real-time changes in oxygen consumption rate (OCR) of three independent experiments in human MDMs treated with ascending doses of PGE2 for 20 h (n=5 biologically independent samples per group; age mean±SE: 47.8±2.105 years). Arrows denote sequential stimulation of cells (i) first with oligomycin (olig), an inhibitor of Complex V ATP generation that allows measurement of basal ATP production; (ii) second with FCCP, an uncoupler of the proton gradient, that allows measurement of maximal respiration; and (iii) third, a combination of rotenone, a Complex I inhibitor, and antimycin A, a Complex III inhibitor (rot/antA), that together block mitochondrial respiration, allowing measurement of non-mitochondrial respiration.

FIG. 1C shows quantification of basal respiration (blue) and extracellular acidification rate (ECAR; red) in human MDMs 20 h following stimulation with PGE2. Data are represented as box and whisker plots (5-95 percentile); one-way ANOVA for basal respiration and ECAR, P<0.0001, Tukey's post-hoc test **P=0.0085, †P=0.0001, #P<0.0001 (n=5 per group; age mean±SE: 47.8±2.105 years).

FIG. 1D shows basal respiration and ECAR in human MDMs 20 h following stimulation with the EP2 agonist butaprost; one-way ANOVA for OCR and ECAR, P<0.0001; Tukey's post-hoc test #P<0.0001 (n=5 per group; age mean±SE: 47.8±2.105 years). See FIG. 5C-5H.

FIG. 1E shows basal respiration and ECAR of peritoneal macrophages isolated from young (3-4 mo) and aged (20-23 mo) Cd11bCre and Cd11bCre;EP2^{lox/lox} mice; two-way ANOVA: effects of age and genotype P<0.0001; Tukey's post-hoc test ****P<0.0001 (n=5 mice per group). See FIGS. 6A-6C.

FIG. 1F shows representative transmission electron microscopy (TEM) images from two independent experiments of peritoneal macrophage mitochondria from young (3-4 mo) and aged (20-23 mo) Cd11bCre (red arrows) and Cd11bCre;EP2^{lox/lox} (blue arrows) mice (scale bar=100 nm; n=6 mice/group).

FIG. 1G shows quantification of numbers, density, percent abnormal, and length to width ratios of mitochondria in young (3-4 mo) and aged (20-23 mo) Cd11bCre and Cd11bCre;EP2^{lox/lox} peritoneal macrophages from TEM imaging. Data are represented as box and whisker plots (5-95 percentile); two-way ANOVA: effects of genotype and age P<0.0001; Tukey's Post-hoc test, ****P<0.0001 (n=250 cells per group).

FIG. 1H shows representative flow-cytometry histograms of peritoneal macrophages from three independent experiments for anti-inflammatory markers CD71 and EGR2 and pro-inflammatory markers CD80 and CD86 from young (3-4 mo) and aged (20-23 mo) Cd11bCre and Cd11bCre;EP2^{lox/lox} mice; n=10,000-20,000 cells per group. See FIG. 6D.

FIG. 1I shows quantification of phagocytosis of fluorescent E. coli particles in peritoneal macrophages isolated from young (3-4 mo) and aged (20-23 mo) Cd11bCre and Cd11bCre;EP2^{lox/lox} mice; two-way ANOVA: effects of age P=0.0147 and genotype P=0.0008; Tukey's post-hoc test *P=0.0127, **P=0.0017 (n=6 mice per group).

FIG. 1J shows hierarchical clustering of significantly regulated immune factors in plasma and hippocampus from young (3-4 mo) and aged (20-23 mo) Cd11bCre and Cd11bCre;EP2^{lox/lox} mice (n=6-10 mice per group). See FIG. 6E-6F.

FIGS. 2A-2I illustrate that myeloid knockdown of the EP2 receptor reverses cognitive aging. Experiments were performed in young (3-4 mo) and aged (20-23 mo) Cd11bCre and Cd11bCre;EP2^{lox/lox} mice.

FIG. 2A is a schematic depicting the Object location memory task (see Methods).

FIG. 2B shows quantification of fraction of time spent exploring the displaced object expressed as % preference for the displaced object (DO); paired t-test *P<0.05, **P<0.01 (n=7-11 mice per group).

FIG. 2C shows representative traces of paths taken to the target hole (green) on the day of testing in the Barnes Maze comparing aged Cd11bCre and Cd11bCre;EP2^{lox/lox} mice.

FIG. 2D shows quantification of percent time in the target quadrant and distance traveled to target hole in the Barnes Maze; ***P<0.001 by two-tailed unpaired Student's t-test (n=7-11 mice per group). See FIG. 7B.

FIG. 2E shows long-term potentiation (LTP) in the CA1 hippocampal region over a 120 minute recording interval. Two-way ANOVA: effects of time and genotype P<0.0001; *P=0.0272 by Sidak's multiple comparisons test with Geisser-Greenhouse correction (n=8 slices, 3 mice per group). See FIG. 7C-7D.

FIG. 2F shows representative immunoblot of two independent experiments of EP2 signaling via the AKT-GSKβ-GYS1 signaling pathway in peritoneal macrophages from 6 mo Cd11bCre and Cd11bCre;EP2^{lox/lox} mice. See FIG. 7E-7H.

FIG. 2G shows glycogen levels of peritoneal macrophages from aged Cd11bCre and Cd11bCre;EP2^{lox/lox} mice. **P<0.01 by two-tailed Student's t-test (n=6 mice per group). See FIG. 7I-7K and FIG. 8.

FIG. 2H shows mice were administered ¹³C-Glucose (1 g/kg by oral gavage) and 4 hours later peritoneal macrophages were isolated for isotope tracing.

FIG. 2I shows quantification of ¹³C-Glucose metabolism in mouse peritoneal macrophages demonstrating shift away from glycogen synthesis and towards glycolysis, pentose phosphate pathway (PPP) and TCA cycle in aged Cd11bCre;EP2^{lox/lox} mice (n=6 mice per group). See FIG. 9A-9C.

FIGS. 3A-J show reduction of glycogen synthesis with EP2 inhibition restores energy metabolism and immune function to youthful levels in human MDMs.

FIG. 3A shows basal respiration from three independent experiments in young (<35 yo) and aged (>65 yo) human MDMs^{+/-}shRNA to GYS1. Effect of age and genotype P<0.0001 by two-way ANOVA; Tukey's post-hoc test *P=0.0225, **P=0.0075, ***P<0.001 (n=5 samples per group). See FIG. 10.

FIG. 3B shows hierarchical clustering of significantly regulated immune factors in young (<35 yo) and aged (>65 yo) human MDMs 20 h after transfection of either scrambled or GYS1 shRNA (n=5-6 biological independent samples per group).

FIG. 3C shows mean fluorescence intensity (MFI) quantification of three independent flow cytometry experiments for anti-inflammatory CD206 and CD163 and pro-inflammatory CD86 and CD64 surface expression in young and aged human MDMs+/−shRNA to GYS1 shows normalization of surface markers to youthful levels. Effect of age and shRNA P<0.0001 by two-way ANOVA; Tukey's post-hoc test *P<0.05, **P<0.01, ***P<0.001, and ****P<0.0001 (n=20,000-40,000 cells per group). See FIG. 10H.

FIG. 3D shows aged (>65 yo) human MDMs were transfected with scrambled or shRNA to GYS1 (shGYS1) and stimulated 8 h later with vehicle or butaprost (100 nm, 20 h). Young human MDMs (<35 yo) received scrambled shRNA+vehicle. Basal respiration and ECAR were measured at 20 h (n=5 biologically independent samples per group; **P<0.01 and ****P<0.0001). See FIG. 10I.

FIG. 3E shows hierarchical clustering of significantly regulated immune factors in supernatant of young (<35 yo) and aged (>65 yo) human MDMs 20 h after transfection with either scrambled or GYS1 shRNA and stimulated with EP2 agonist butaprost or vehicle 8 h later, as in (FIG. 3D); n=5-6 biological independent samples per group. Rescue of immune phenotypes in aged human MDMs+GYS1 shRNA is refractory to further EP2 receptor activation with butaprost.

FIG. 3F shows Fold change of glycogen levels in young and aged human MDMs+/−EP2 inhibitor C52 (100 nM, 20 h). Effects of age and treatment P<0.0001 by two-way ANOVA; Tukey's post-hoc test ****P<0.0001 (n=6 samples per group). See FIG. 11B-11C.

FIG. 3G shows quantification of basal respiration and ECAR in young and aged human MDMs+/−C52 (100 nM, 20 h). Effects of age and treatment P<0.0001 by two-way ANOVA; Tukey's post-hoc test ****P<0.0001 (n=5 samples per group). See FIG. 11D

FIG. 3H shows quantification of mitochondrial protein levels in young and aged human MDMs+/−C52 (100 nM, 20 h); *P<0.05, **P<0.01, ***P<0.001, ****P<0.0001 by two-way ANOVA with Tukey's post-hoc test (n=6 samples per group). See FIG. 11E-11G.

FIG. 3I shows LC-MS targeted metabolomics of glycolysis, pentose phosphate pathway, and TCA cycle intermediates in young and aged human MDMs+/−C52 (100 nM, 20 h), n=3 samples per group. See FIG. 11I.

FIG. 3J shows quantification of phagocytosis of fluorescent E. coli particles in young and aged human MDMs+/−C52 (100 nM, 20 h; n=6 samples per group). ***P=0.0006, ****P<0.0001 by two-way ANOVA with Tukey's multiple comparisons tests.

FIGS. 4A-4M show inhibition of EP2 signaling in aged mice reverses age-associated inflammation and spatial memory loss.

FIG. 4A shows young (3-4 mo) and aged (22-24 mo) mice were treated with vehicle or C52 (10 mg/kg/d, 1 mo). Hierarchical clustering of significantly regulated cytokines and chemokines in plasma of young and aged mice+/−C52 (n=3-4 per group; FIG. 14A).

FIG. 4B shows hierarchical clustering of significantly regulated cytokines and chemokines in hippocampi of young and aged mice+/−C52 (10 mg/kg/d, 1 mo; n=3-4 per group; FIG. 14B).

FIG. 4C shows representative immunofluorescent staining of hippocampal CA1 regions of microglial activation in aged mice+/−C52 (10 mg/kg/d, 1 mo; 630× magnification, scale bar=10 μm; n=8 slices in 4 mice per group, see FIGS. 14C-14D and 17A-17B.

FIG. 4D shows mice treated+/−C52 (10 mg/kg/d, 10 days) were administered U-13C-Glucose at day 10 (1 g/kg by oral gavage), and brain microglia (and peritoneal macrophages, see FIG. 15A-15C) were harvested 4 h later. U-13C-Glucose and labeled fractions were measured by LC/MS. Aged microglia show increased incorporation of ¹³C-Glucose in glycogen precursors and reduced incorporation in glycolytic TCA intermediates. Inhibition of EP2 restores glycogen precursors and glycolytic/TCA cycle intermediates to youthful levels. See FIG. 17C.

FIG. 4E shows quantification of percent preference in the novel displacement object task in young and aged mice+/−C52 (10 mg/kg/d, 2 weeks). **P=0.0014, ***P=0.0003, ****P<0.0001 by paired t-test (n=8-9 mice per group; see FIG. 16B).

FIG. 4F shows quantification of distance traveled to target hole in the Barnes maze. Effect of age P<0.0001, effect of treatment P=0.0009 by two-way ANOVA; Tukey's post-hoc test ****P<0.0001 (10 mg/kg/d, 2 weeks; n=10 mice per group). See FIG. 15D.

FIG. 4G shows LTP deficits in aged mice are rescued with C52 treatment. Effect of age and treatment P<0.0001 by two-way ANOVA; **P=0.0096 by Sidak's multiple comparisons test with Geisser-Greenhouse correction (10 mg/kg/d, 1 mo; n=10-12 slices, 5 mice per group). Young-veh vs. Young-052 treatment: P=0.1840 by Sidak's multiple comparisons test with Geisser-Greenhouse correction. See FIG. 15E.

FIG. 4H shows synaptic mitochondria were isolated from synaptosome fractions from 16 mo mice treated+/−C52 (10 mg/kg/d, 10 days). Coupling between mitochondrial ETC and oxidative phosphorylation (using succinate as the substrate) was determined using the Seahorse XFe24 analyzer to quantify the Respiration Control Ratio (RCR, or ratio of State III/State IVo), a measure of mitochondrial integrity; **P=0.01, two-tailed Student's t-test (n=7 mice per group). See FIG. 15F-15G.

FIG. 4I shows hierarchical clustering of significantly regulated cytokines and chemokines in plasma (top) and hippocampus (bottom) of young (3-4 mo) and aged (20-22 mo) mice+/−brain impermeant PF-04418948 (PF or veh; 2.5 mg/kg/d by gavage, 6 weeks; n=3-5 per group). See FIG. 18A-18C.

FIG. 4J shows quantification of percent preference in the novel displacement object task in young (3-4 mo) and aged (20-22 mo) mice+/−PF-04418948 (PF; 2.5 mg/kg/d, 6 weeks). **P=0.0074, ***P=0.0004, ****P<0.0001 by paired t-test (n=6-7 mice per group).

FIG. 4K shows quantification of primary latency to target hole in the Barnes Maze+/−PF-04418948 (2.5 mg/kg/d, 6 weeks). Effects of age (P=0.0008) and treatment (P=0.0034) by two-way ANOVA; Tukey's post-hoc test ***P=0.0005, **P=0.0022 (n=6-7 mice per group). See FIG. 18D-18E

FIG. 4L shows LTP deficits in aged mice are rescued with PF-04418948 treatment (2.5 mg/kg/d, 6 weeks). Effect of age and treatment P<0.0001 by two-way ANOVA; ***P=0.00698 by Sidak's multiple comparisons test with Geisser-Greenhouse correction (2.5 mg/kg/d, 1.5 mo; n=6-8 slices, 5 mice per group). See FIG. 18F.

FIG. 4M shows principal component analysis of transcriptomics of peritoneal macrophages derived from young (3-4 mo) and aged (20-22 mo) mice from (FIG. 4K). Ellipses represent 95% Cl. See FIG. 19-20

FIGS. 5A-5H show PGE2 regulates macrophage energy metabolism via the EP2 receptor in human macrophages.

FIG. 5A shows the PGE2 synthetic pathway. Arachidonic acid is converted to the prostaglandin precursor PGH2 by the action of constitutive COX-1 and inducible COX-2; this step is inhibited by non-steroidal anti-inflammatory drugs (NSAIDs). PGE2 synthase converts PGH2 to PGE2 which can bind to four distinct G-protein coupled receptors (E-prostanoid receptors, or EP1-4 receptors).

FIG. 5B shows (Top): Representative immunoblot of two independent experiments quantifying COX-2 and PGE2 synthase in young and aged human MDMs. (Bottom) Quantification demonstrates a significant upregulation of synthetic enzymes COX-2 and mPGE2 synthase in aged (>65 years) human MDMs compared to young (<35 years) (n=6 biologically independent samples per group; **P=0.030, *P=0.0106, Student's two tailed t-test).

FIG. 5C shows (Top): Representative immunoblot of two independent experiments quantifying EP receptors 1-4 in young and aged human MDMs; last lane, HEK cells transfected with EP receptor cDNA as positive control. (Bottom): Quantification demonstrates selective upregulation of the EP2 receptor in aged (>65 years) human MDMs compared to young (<35 years; n=6 biologically independent samples per group; ****P<0.0001, Student's two tailed t-test).

FIG. 5D shows quantification of basal respiration and ECAR of human MDMs treated with ascending doses of the EP1 agonist iloprost at 20 h (n=5 biologically independent samples per group).

FIG. 5E shows quantification of basal respiration and ECAR of human MDMs treated with ascending doses of the EP3 agonist AE248 at 20 h (n=5 biologically independent samples per group).

FIG. 5F shows quantification of basal respiration and ECAR of human MDMs treated with ascending doses of the EP4 agonist AE1329 at 20 h (n=5 biologically independent samples per group).

FIG. 5G shows a dose response of the brain-impermeant EP2 antagonist PF04418948 at 20 h. Data are represented as box and whisker plots (5-95 percentile); one-way ANOVA for OCR and ECAR, P<0.0001; Tukey's post-hoc test **P=0.0083, ***P=0.0005, #P<0.0001 (n=5 biologically independent samples per group).

FIG. 5H shows a dose response of the brain-penetrant EP2 antagonist C52 at 20 h. Data are represented as box and whisker plots (5-95 percentile); one-way ANOVA for OCR and ECAR, P<0.0001; Tukey's post-hoc test #P<0.0001 (n=5 biologically independent samples per group).

FIGS. 6A-6F show PGE2 synthesis is upregulated in aged mouse macrophages and brain; myeloid knockdown of the EP2 receptor restores youthful immune state in aged Cd11bCre;EP2^{lox/lox} mice.

FIG. 6A shows the LC/MS quantification of PGE2 levels in plasma and cerebral cortex of aged (18-20 mo) and young mice (3-4 mo). ****P<0.0001 by two-tailed Student's t-test (n=6 mice per group)

FIG. 6B shows aging does not alter cerebral cortex levels of the other prostanoids, including PGD2, PGF2α, and prostacyclin (measured by 6-keto-PGF1a; n=6 mice per group).

FIG. 6C shows (Top): Representative immunoblot of EP1-EP4 receptor levels in mouse peritoneal macrophages isolated from young (3-4 mo) and aged (18-20 mo) mice; last lane, HEK cells transfected with EP receptor cDNAs as positive control (n=6 mice per group). (Bottom): Quantification reveals that the EP2 receptor is selectively upregulated in aged mouse peritoneal macrophages, similar to human MDMs; **P=0.0018 (n=6 mice per group).

FIG. 6D shows mean fluorescence intensity (MFI) quantification from three independent experiments for the anti-inflammatory markers CD71 and EGR2 and proinflammatory markers CD80 and CD86 in young (3-4 mo) and aged (20-23 mo) Cd11bCre and Cd11bCre;EP2^{lox/lox} mice, n=10,000-20,000 cells per group. Two-way ANOVA: effects of genotype and age P<0.0001; Tukey's post-hoc test **P<0.01, ***P<0.001, ****P<0.0001.

FIG. 6E shows quantification of immune factors in plasma comparing aged Cd11bCre and aged Cd11bCre;EP2^{lox/lox} mice; *P<0.05, **P<0.01 by two-tailed Student's t-test (n=6 mice per group).

FIG. 6F shows quantification of immune factors in hippocampi comparing aged Cd11bCre and aged Cd11bCre;EP2^{lox/lox} mice; *P<0.05, **P<0.01, ***P<0.001 by two-tailed Student's t-test (n=6 mice per group).

FIGS. 7A-7K show myeloid knockdown of the EP2 receptor rescues age-associated spatial memory deficits and inhibits glycogenesis in an AKT-GSK3β-GYS1 dependent manner.

FIG. 7A: shows representative immunoblot and quantification from two independent experiments comparing EP2 levels in 6 mo Cd11bCre and WT mice (n=6 per group).

FIG. 7B shows primary latency in the Barnes Maze for the five learning trials.

7C shows representative immunoblot and quantification of hippocampal pre-synaptic proteins synapsin and SNAP-25 and post-synaptic proteins PSD95 and CamKIIa; ***P<0.001, ****P<0.0001 by two-tailed Student's t-test (n=6 mice per group).

FIG. 7D shows input/output curves as a measure of basal synaptic transmission in the CA1 region of the hippocampus (n=8 slices, 3 mice per group).

FIG. 7E shows PGE2 activation of the EP2 receptor activates AKT signaling through phosphorylation of Ser473. Activated pAKT (Ser473) inactivates GSK3β through inhibitory phosphorylation of Ser9. Inactivation of GSK3β leads to constitutive activity of glycogen synthase 1 (GYS1) and glycogen synthesis. Conversely, deletion or antagonism of EP2 receptor signaling leads to downstream inhibitory phosphorylation of Ser641, 645, and 649 on GYS1 by activated, non-phosphorylated GSK3β.

FIG. 7F shows quantification of EP2, activated pAKT (Ser 473)/total AKT, and inactivated pGSK3β (Ser 9)/total GSK3β levels in peritoneal macrophages from 6 mo Cd11bCre and Cd11bCre;EP2^{lox/lox} mice, n=6 mice per group; ****P<0.0001 by two-tailed Student's t-test.

FIG. 7G shows representative immunoblots and quantification of the EP2-AKT-GSK3β-GYS1 signaling pathway in mouse peritoneal macrophages isolated from 6 month old wild type C57B6/J mice treated with EP2 antagonist C52 (100 nM), EP2 agonist butaprost (100 nM), or PGE2 (100 nM) for 20 h.

FIG. 7H shows quantification of (G); P<0.0001 by one-way ANOVA; Tukey's post-hoc test **P<0.01, ***P<0.001, and ****P<0.0001 (n=6 mice per group).

FIG. 7I shows quantification of glycogen levels in human MDMs (age mean±SE: 43.9±3.451 years) treated with EP2 agonist butaprost (100 nM) or EP2 antagonist C52 (100 nM) for 20 h. P<0.001 by one-way ANOVA; Tukey's post-hoc test ****P<0.0001 (n=9 per group).

FIG. 7J shows untargeted liquid chromatography-mass spectroscopy (LC-MS) analysis of human MDMs (age mean+/−SE: 43.9+/−3.451 years) treated with C52 (100 nM, 20 h) demonstrates upregulation of proximal glucose metabolites in the glycolytic pathway (G-6P and F-6P) and down regulation of the redox metabolite glutathione (GSSG). Red circles represent metabolites with fold change>1.5, blue circles with fold change<1.5; q-value<0.05 with FDR correction (n=6 biologically independent samples per group).

FIG. 7K shows enrichment pathway analysis of (J).

FIGS. 8A-8H show the effects of PGE2, the EP2 agonist butaprost, and EP2 inhibitor C52 in human MDMs. Human MDMs are from n=6 donors (age mean±SE: 43±8.344 years)

FIG. 8A: shows representative immunoblots and quantification of two independent experiments measuring effects of ascending doses of PGE2 (A) and the EP2 agonist butaprost (B) at 20 h on Ser473 pAKT/total AKT in human MDMs. P<0.0001 by one-way ANOVA; Tukey's post-hoc test *P=0.0340, ***P=0.006, #P<0.0001 (n=6 samples per group).

FIG. 8C-8D show representative immunoblots and quantification of two independent experiments measuring the effect of ascending doses of PGE2 (C) and the EP2 agonist butaprost (D) at 20 h on Ser9 pGSK3β/total GSK3β in human MDMs. P<0.0001 by one-way ANOVA; Tukey's post-hoc test #P<0.0001 (n=6 samples per group).

FIG. 8E-8G show representative immunoblots and quantification of time course of pAKT (Ser473)/total AKT (E), pGSK3β (Ser9)/total GSK31 (F), and pGYS1 (Ser641, 645, 649)/total GYS1 (G) from six independent experiments in huMDMs treated with butaprost (100 nM, red) or C52 (100 nM, blue) from 0 h to 20 h. P<0.0001 by one-way ANOVA (n=12 samples per group).

FIG. 8H shows representative immunoblot and quantification of two independent experiments measuring effect of butaprost (20 h, 100 nM) and the C52 (20 h, 100 nM) on EP2 levels in human MDMs (n=6 samples per group).

FIGS. 9A-9E show macrophage EP2 signaling regulates proximal glucose metabolism and increases glycogen synthesis Human MDMs are from donors (age mean±SE: 42.13±3.674 years)

FIG. 9A shows a schematic depicting U-13C-glucose metabolism via Glucose-1P (G1P) and UDP-glucose to glycogen synthesis (yellow shaded box) versus flux towards the pentose phosphate pathway, glycolysis, and the citric acid cycle with associated mass-labeled molecules.

FIG. 9B shows schematic depicting changes in glucose metabolism in FIG. 2H-I.

FIG. 9C shows isotope tracing of U-13C-Glucose metabolism was performed in human MDMs treated with the EP2 agonist butaprost (100 nM, 20 h) or the EP2 inhibitor C52 (100 nM, 20 h; n=6 independent samples per group). Activation of EP2 signaling with butaprost increases incorporation of mass labeling in glycogen precursors G-1P and UDP-glucose (increases glycogen synthesis) and reduces glycolysis (reduced mass labeling of F-1,6-BP and pyruvate) and TCA cycle intermediates (citrate and succinate); inhibition of EP2 with C52 conversely reduces glycogen synthesis and increases glycolysis and TCA cycle.

FIG. 9D shows representative flow-cytometry histograms and corresponding MFI quantification of huMDMs+/−the EP2 inhibitor C52 (100 nM, 20 h) from three independent experiments. Surface levels of anti-inflammatory markers CD206 and CD163 increase with inhibition of EP2 signaling while levels of proinflammatory markers CD86 and CD64 decrease, indicating a shift towards an anti-inflammatory polarization state. **P<0.01, ***P=0.002 by two-tailed Student's t-test (n=20,000-40,000 cells per group).

FIG. 9E shows quantification of phagocytosis of fluorescent E. coli particles in human MDMs treated with EP2 inhibitor C52 (100 nM, 20 h) from two independent experiments. ***P=0.0002 by two-tailed Student's t-test (n=9 samples per group).

FIGS. 10A-10I show knockdown of GYS1 increases macrophage energy metabolism and anti-inflammatory polarization state. (10A-10F) Human MDMs are from donors (age mean±SE: 47.2±1.582 years); (10G-10I) Human MDMs are from young (<35 years) and aged (>65 years) donors.

FIG. 10A shows representative immunoblots and quantification of human MDMs transfected with two different shRNAs to human GYS1 at 8 h. P<0.0001 by one-way ANOVA; Tukey's post-hoc test ****P<0.0001 (n=6 biologically independent samples per group).

FIG. 10B shows quantification of glycogen levels in human MDMs transfected with shRNAs to GYS1 at 8 h. P<0.0001 by one-way ANOVA; Tukey's post-hoc test ****P<0.0001 (n=6 biologically independent samples per group).

FIG. 10C shows representative traces and quantification of OCR and ECAR for three independent experiments in human MDMs transfected with shRNAs for GYS1 at 8 h (n=5 samples per group). P<0.0001 by one-way ANOVA; Tukey's post-hoc test ****P<0.0001 (n=6 biologically independent samples per group).

FIG. 10D shows hierarchical clustering of targeted metabolomics for glycolysis, pentose phosphate shunt, and citric acid cycle metabolites in human MDMs transfected with shRNA to GYS1 at 8 h (n=5 samples per group).

FIG. 10E shows isotope tracing of U-13C-Glucose in human MDMs transfected with shRNA to GYS1 at 8 h reveals a decreased labeling in the glycogen precursor UDP-glucose and an increase in glycolytic intermediates F-1,6BP and pyruvate (n=6 samples per group).

FIG. 10F shows representative flow-cytometry histograms of three independent experiments for the proinflammatory markers CD86 and CD64 and anti-inflammatory markers CD206 and CD163 in human MDMs+/−shRNA to GYS1.

FIG. 10G shows representative trace of real-time changes in OCR from three independent experiments of young (age<35 yo) and aged (>65 yo) human MDMs+/−shRNA to GYS1 (n=5 samples per group).

FIG. 10H shows representative histograms of anti- and pro-inflammatory surface markers in young and aged human MDMs+/−shRNA to GYS1.

FIG. 10I shows representative traces of real-time changes in oxygen consumption rate (OCR) of two independent experiments in young (<35 yo) and aged (>65 yo) human MDMs nucleofected with shRNA to GYS1 (shGYS1). 8 h post nucleofection cells were treated with butaprost (100 nm, 20 h). Oxygen consumption rate (OCR) in GYS1-deficient aged human MDMs is refractory to treatment with EP2 agonist butaprost (100 nM, 20 h).

FIGS. 11A-11K show inhibition of EP2 signaling with C52 restores youthful AKT/GSK3β/GYS1 signaling, mitochondrial respiration and lower glycogenesis.

FIG. 11A: shows quantification of PGE2 levels in young (<35 years) and aged (>65 years) human MDMs+/−C52 (100 nM, 20 h). Effects of age and treatment P<0.0001 by two-way ANOVA; Tukey's post-hoc test ****P<0.0001 (n=5 samples per group).

FIG. 11B shows human MDMs were derived from young (<35 yo) and aged (>65 yo) blood. Representative immunoblots of the EP2 signaling cascade in young and aged human MDMs+/−C52 (100 nM, 20 h).

FIG. 11C shows quantification of effects of age and C52 treatment in (A). P<0.0001; Tukey's post-hoc test **P=0.002, ***P=0.0005, ****P<0.0001 (n=6 samples per group).

FIG. 11D shows representative traces of real-time changes in OCR from three independent experiments of young and aged human MDMs+/−C52(100 nM, 20 h; n=5 samples per group).

FIG. 11E shows representative immunoblots of effects of EP2 inhibition on mitochondrial protein levels in young and aged huMDMs (n=6 biologically independent samples per group; OMM=outer mitochondrial membrane, IMM=inner mitochondrial membrane). See FIG. 3H.

FIG. 11F shows quantification of membrane potential (TMRE) in young and aged human MDMs+/−EP2 inhibitor C52 (100 nM, 20 h). Effects of age P=0.0009 and treatment P=0.0333 by two-way ANOVA; Tukey's post-hoc test ***P=0.0006, **P=0.0088 (n=6 samples per group).

FIG. 11G shows quantification of reactive oxygen species (ROS) in young and aged human MDMs+/−EP2 inhibitor C52 (100 nM, 20 h). Effects of age P=0.0022 and treatment P=0.0055 by two-way ANOVA; Tukey's post-hoc test **P=0.0045, ##P=0.0084.

FIG. 11H shows young and aged human MDMs were incubated with U-¹³C-Glucose for 20 h+/−butaprost or compound 52. Aged human MDMs demonstrated increased labeling in glycogen precursors (G1P and UDP-Glucose) and decreased labeling in glycolytic and TCA intermediates. This was prevented with EP2 inhibition. F6P: fructose 6-phosphate, G3P: glyceraldehyde 3-phosphate, KG: alpha-ketoglutarate; N=3 biologically independent samples per group.

FIG. 11I shows quantification of TCA metabolites from FIG. 3I. Note normalization of citrate, a-KG, succinate, fumarate, and malate in aged huMDMs with EP2 inhibition. Also note that itaconate, which is increased in models of acute macrophage stimulation with LPS, is not changed with aging (n=3 biologically independent samples/group; 2 way ANOVA with Tukey multiple comparisons; *P<0.05, **P<0.01, ***P<0.001). See FIG. 12.

FIG. 11J shows representative traces of real-time changes in oxygen consumption rate (OCR) of two independent experiments in peritoneal macrophages isolated from young (3-4 mo) and aged (1820 mo) mice treated with COX-2 inhibitor SC236 (100 nM, 20 h).

FIG. 11K shows quantification of basal respiration and ECAR reveals inhibition of COX-2 partially phenocopies EP2 receptor antagonism in aged (18-20 mo) mouse peritoneal macrophages. Effects of age and treatment P<0.0001 and P=0.004 by two-way ANOVA, respectively; ****P<0.0001 by Tukey's posthoc test (n=5 samples per group).

FIGS. 12A-12D show EP2 blockade does not alter LPS-mediated glucose metabolism reprogramming.

FIG. 12A shows human MDMs+/−LPS (100 ng/mL, 20 h; n=4-6 biologically independent samples per group, age mean±SE: 42.3±8.212 years) were co-stimulated with C52 (100 nM, 20 h). Blockade of EP2 does not rescue OCR and ECAR in LPS-treated human MDMs

FIG. 12B shows ¹³C-Glucose isotope tracing of human MDMs+/−LPS (100 ng/mL, 20 h)+/−C52 (100 nM, 20 h) demonstrates reprogramming of the TCA cycle towards increased production of itaconate with LPS stimulation that is not reversed with EP2 inhibition (n=3 biologically independent samples per group).

FIG. 12C shows hierarchical clustering of targeted metabolomics of glucose and TCA metabolites demonstrates that LPS treated human MDMs clusters with LPS+C52 human MDMs (n=3 per group).

FIG. 12D shows model highlighting differences in glucose metabolism and TCA in LPS-stimulated macrophages and aged macrophages. LPS stimulation upregulates glycolysis and lactate production through increased itaconate production from aconitate; increased itaconate suppresses SDH. In aged human MDMs, glucose is diverted from glycolysis to glycogen, reducing glucose flux into the TCA cycle; aging is characterized by low SDH activity arising from deficient Sirt3 deacetylation of Complex II subunits as a result of declining NAD+ levels. In both cases, lower succinate dehydrogenase activity leads to accumulation of the pro-inflammatory TCA intermediate succinate which enhances pro-inflammatory gene expression by stabilizing the transcription factor HIF1α.

FIGS. 13A-13G show aged myeloid cells metabolize glucose but not alternate fuel substrates like glutamine, pyruvate, and lactate.

FIG. 13A shows a diagram illustrating fuel substrates for TCA and OXPHOS that were tested with mass labeling in (13B-13E). Glucose and pyruvate/lactate can feed into the TCA via acetyl-CoA; glutamine is metabolized via glutaminolysis to α-ketoglutarate, a TCA intermediate.

FIG. 13B shows young (<35 yo) and aged (>65 yo) human MDMs received U-¹³C-Glucose [M+6] for 20 h. Isotope tracing reveals young macrophages can incorporate significantly higher ¹³C from glucose into the citric acid cycle (TCA) compared to aged macrophages (P=0.0026 by Welch's t-test for ¹³C citrate[M+2] proportion in young versus aged cells; n=3 samples per group).

FIG. 13C shows young (<35 yo) and aged (>65 yo) human MDMs received U-¹³C-Pyruvate [M+3] for 20 h. Isotope tracing reveals young macrophages are able to incorporate significantly higher ¹³C from pyruvate into the citric acid cycle (TCA) compared to aged macrophages (P<0.0001 by Welch's t-test for ¹³C-citrate[M+2] in young versus aged cells; n=3 samples per group).

FIG. 13D shows young (<35 yo) and aged (>65 yo) human MDMs received U-13C—Lactate [M+3] for 20 h. Isotope tracing reveals young macrophages are able to incorporate significantly higher ¹³C from lactate into the citric acid cycle (TCA) than aged macrophages (P=0.0083 by Welch's t-test for ¹³C citrate[M+2] in young versus aged cells; n=3 samples per group).

FIG. 13E shows young (<35 yo) and aged (>65 yo) human MDMs received U-¹³C-Glutamine [M+5] for 20 h. Isotope tracing reveals young macrophages are able to incorporate significantly higher ¹³C from glutamine into the citric acid cycle (TCA) than aged macrophages (P<0.0001 by Welch's t-test for ¹³C-αKetoglutarate [M+5] in young versus aged cells; n=3 samples per group).

FIG. 13F shows representative trace of real-time changes in OCR from two independent experiments demonstrating increased dependence on glucose and reduced capacity by aged mouse macrophages (20-23 mo) for oxidative phosphorylation (n=5 samples per group). UK5099: mitochondrial pyruvate carrier inhibitor; BPTES: glutaminase inhibitor; etomoxir: inhibitor of carnitine palmitoyltransferase 1 (CPT1) which transports fatty acids into mitochondrial matrix. For dependency traces cells received UK5099 in first injection and BPTES/Etomoxir in second injection as indicated on figure. For capacity traces cells received BPTES/Etomoxir in first injection and UK5099 in second injection.

FIG. 13G shows quantification of (F); n=5 samples per group; ****P<0.0001.

FIGS. 14A-14F show the effects of EP2 inhibition in young and aged mice on inflammation.

FIG. 14A shows the quantification of immune factors in plasma from young (3-4 mo) and aged (22-24 mo) mice+/−C52 (10 mg/kg/d for 1 mo; n=3-4 mice per group) shows restoration of aged immune factor levels to youthful levels with EP2 antagonism.

FIG. 14B shows quantification of immune factors in hippocampi from young (3-4 mo) and aged (22-24 mo) mice+/−C52 (10 mg/kg/d for 1 mo; n=3-4 mice per group) shows restoration of aged immune factor levels to youthful levels with EP2 antagonism.

FIG. 14C shows quantification of percent CD68+/Iba1+ cells in CA3 hippocampus from young and aged mice+/−C52 (10 mg/kg/day, 1 mo); effect of age and treatment P<0.0001 by two-way ANOVA; Tukey's post-hoc test ****P<0.0001 (n=8 slices, 3-4 mice per group).

FIG. 14D shows quantification of microglial numbers in (C).

FIG. 14E shows representative immunoblot of pre-synaptic and post-synaptic proteins synapsin, PSD95, SNAP25, CamKIIa in young and aged mice+/−C52 (10 mg/kg/day, 1 mo) FIG. 14F shows quantification of (E). Synapsin: effects of age (P=0.0685) and treatment (P=0.0072) by two-way ANOVA; Tukey's post-hoct test **P=0.0069, ***P=0.0010. PSD95: effects of age (P=0.0019) and treatment (P=0.0009) by two-way ANOVA; Tukey's post-hoc test **P=0.0020, ***P=0.0007. SNAP25: effects of age (P=0.1930) and treatment (P=0.0463) by two-way ANOVA; Tukey's post-hoc test *P=0.0121. CamKIIa: effects of age (P=0.9210) and treatment (P=0.0025) by two-way ANOVA; Tukey's post-hoc test *P=0.0132.

FIGS. 15A-15G show the effects of in vivo EP2 inhibition on macrophage metabolism and synaptic mitochondrial OCR in young and aged mice.

FIG. 15A shows young and aged mice were administered C52 for 10 days at 10 mg/kg/day by oral gavage. On day 10, U-¹³C-Glucose (1 g/kg by gavage) was administered for in vivo isotope tracing of brain microglia and peritoneal macrophages harvested 4 hours later (n=6 mice per group).

FIG. 15B shows gating strategy for isolation of CD45midCd11b+ microglia from young (3-4 mo) and aged (22-24 mo) mice

FIG. 15C shows peritoneal macrophage metabolite labeling following in vivo U-¹³C-Glucose isotope tracing (n=6 mice per group).

FIG. 15D shows quantification of percent time in the target quadrant of the Barnes maze. Effect of age P=0.0023, effect of treatment P=0.0002 by two-way ANOVA; Tukey's post-hoc test ***P=0.0001, ****P<0.0001.

FIG. 15E shows long-term potentiation (LTP) in the CA1 hippocampal region over a 120 minute recording interval. Acute administration of C52 (100 nM, 1 hr) prior to theta-burst stimulation (TBS) does not alter LTP in aged mice (20-22 mo mice, n=3 mice per group).

FIG. 15F shows synaptic mitochondria were isolated from synaptosome fractions prepared from 16 mo aged mice treated+/−C52 (10 mg/kg/d, 10 days). Coupling between mitochondrial ETC and oxidative phosphorylation (using succinate as the substrate) was determined using the Seahorse XFe24 analyzer. *P<0.05, two-tailed Student's t-test (n=7 mice per group).

FIG. 15G shows coupling assay trace of synaptic mitochondria from aged mice+/−C52 for 10 days. Rates of basal complex II respiration as well as states III (ADP stimulated respiration), IV (oligomycin) and Illu (FCCP) were consecutively measured (n=7 mice per treatment group).

FIGS. 16A-16E show average speed and % Time within the inner zone of the novel object location chamber; lack of EP2-mediated bioenergetic effects in primary neurons and astrocytes.

FIG. 16A shows average speed (inches/second) and % Time Inner Zone in young (3-4 mo) and aged (20-23 mo) Cd11bCre;EP2^{lox/lox} mice.

FIG. 16B shows average speed (inches/second) and % Time Inner Zone in young (3-4 mo) and aged (22-24 mo) mice treated with C52 compound (10 mg/kg/d, 1 mo).

FIG. 16C shows average speed (inches/second) and % Time Inner Zone in young (3-4 mo) and aged (20-22 mo) mice treated with PF compound (2.5 mg/kg/d, 6 weeks.)

FIG. 16D shows representative traces of real-time changes in oxygen consumption rate (OCR) of three independent experiments on mouse hippocampal neurons treated with PGE2 (100 nM, 20 h), Butaprost (100 nM, 20 h), and C52 (100 nM, 20 h). (n=6-7 biologically independent samples per group) and quantification of OCR and ECAR. There are no significant effects of EP2 agonist or antagonist on primary neurons.

FIG. 16E shows representative trace of real-time changes in oxygen consumption rate (OCR) of three independent experiments on mouse astrocytes treated with PGE2 (100 nM, 20 h), Butaprost (100 nM, 20 h), and C52 (100 nM, 20 h). (n=7 biologically independent samples per group) and quantification of OCR and ECAR. There are no significant effects of EP2 agonist or antagonist on primary astrocytes.

FIGS. 17A-17C show EP2 receptor expression in brain microglia and TEM of microglial mitochondria in C52 treated young and aged mice.

FIG. 17A shows immunoblot of EP2 from isolated microglia in young (3-4 mo) and aged (20-22 mo) mice (n=6 mice per group).

FIG. 17B shows representative immunofluorescent staining in aged (20-22 mo) mice of hippocampal CA1 region reveals EP2 expression in Iba1+ microglia (n=6 mice per group). Scale bar=10 μm

FIG. 17C shows transmission electron microscopy (TEM) images at 5000× of microglia in the CA3 region of the hippocampus from young (3-4 mo) and aged (22-24 mo) mice+/−C52 (10 mg/kg/d, 1 month). Aged mice harbor abnormal, non-electron dense mitochondria; these features are rescued with C52 treatment. Arrows (black for vehicle, white for C52) point to mitochondria within microglia; blue shaded areas are non-microglial cells. Nu=Nuclei; white scale bars=1 μm.

FIGS. 18A-18F show the effects of peripheral EP2 antagonism with non-brain penetrant EP2 inhibitor. Young (3-4 mo) and aged mice (20-22 mo) were treated with vehicle or PF-04418948 at 2.5 mg/kg/d for 6 weeks.

FIG. 18A shows LC/MS analysis of plasma and brain levels of PF-04418948 (2.5 mg/kg/d, 6 weeks). PF-04418948 was not detected in whole brain lysates of treated mice (n=5-6 mice per group).

FIG. 18B shows quantification of significantly regulated immune factors in hippocampi (n=3-5 mice per group).

FIG. 18C shows quantification of significantly regulated immune factors in plasma (n=3-5 mice per group).

FIG. 18D shows primary latency in the Barnes Maze for the five learning trials.

FIG. 18E shows representative traces of paths taken to the target hole (green) on the day of testing in the Barnes Maze comparing aged mice+/−PF-04418948. 18F Input/output curves as a measure of basal synaptic transmission in the CA1 region of the hippocampus (n=8 slices, 3 mice per group).

FIGS. 19A-19E show transcriptomics of primary peritoneal macrophages from young and aged mice+/−the non-brain penetrant EP2 inhibitor PF-04418948

FIG. 19A shows a heatmap of 1449 genes by nanostring reveals aged mice treated with PF-04418948 cluster with young mice treated with vehicle or PF-04418948.

FIG. 19B shows a volcano plot of peritoneal macrophages harvested from aged versus young vehicle treated mice. Red dots indicate genes that are absolute-value[log 2(FC)] 2 and FDR<0.05 by t test with Benjamini-Hochberg correction.

FIG. 19C shows top 10 signaling pathways from the Reactome pathway database for FDR<0.05 metabolites comparing young vs aged mice. Volcano plot of peritoneal macrophages harvested from aged+PF-04418948 versus aged+vehicle treated mice. Red dots indicate genes that are absolute-value[log 2(FC)] 1.5 and FDR<0.05 by t-test with Benjamini-Hochberg correction. Hierarchical clustering of top differentially regulated chemokine and cytokines transcripts (FDR<0.05) demonstrates that PF-04418948 treatment shifts expression towards young macrophage levels.

FIG. 20 shows PF-04418948 treatment restores expression levels of glycolytic and TCA genes in aged mice. Nanostring analysis of significantly (FDR<0.05) demonstrated differential expression of bioenergetic transcripts in peritoneal macrophages isolated from young (3-4 mo) and aged (20-22 mo) mice+/−PF-04418948 (2.5 mg/kg/d for 6 weeks). Aged peritoneal macrophages exhibit suppressed gene expression encoding critical glycolytic enzymes, including the rate-limiting enzyme phosphofructokinase-1 (Pfk-1) as well as the rate-setting citric acid cycle enzyme, citrate synthase. Peripheral myeloid EP2 inhibition with PF-04418948 corrects the age-associated suppression of myeloid glycolytic and TCA gene expression.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, medicine, neurology, and the like, which are within the skill of the art.

The following examples are put forth so as to provide those of ordinary skill in the art with a complete disclosure and description of how to perform the methods and use the compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary.

It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S.

Patent law and the term is open-ended, allowing for the presence of more than that which is recited so long as basic or novel characteristics of that which is recited is not changed by the presence of more than that which is recited, but excludes prior art embodiments.

### Definitions

The term “antagonist” or “inhibitor,” refers to a modulator that, when contacted with a molecule of interest, causes a decrease in the magnitude of a certain activity or function of the molecule compared to the magnitude of the activity or function observed in the absence of the antagonist.

The terms “cognitive disorders (CDs)” and “cognitive decline”, also known as neurocognitive disorders (NCDs), as used herein refer to a category of mental health disorders that primarily affect cognitive abilities including, learning, memory, perception, and problem-solving. Neurocognitive disorders include delirium, attention deficit disorder, schizophrenia and mild and major neurocognitive disorder (previously known as dementia).

Cognitive disorders are deficits in cognitive ability (acquired rather than developed) that typically decline over time and may have underlying pathology in the brain. The DSM-5 defines six key domains of cognitive function: executive function, learning and memory, perceptual-motor function, language, complex attention, and social cognition.

Causes vary between the different types of disorders but, most include damage to the memory portions of the brain. Treatments depend on how the disorder began. Medication and therapies are the most common treatments; however, for some types of disorders, such as certain types of amnesia, treatments can suppress the symptoms, but there is currently no cure.

The term “small molecule” as used herein refers to an organic compound, including an organometallic compound, of a molecular weight less than about 3 kDa, that is not a polynucleotide, a polypeptide, a polysaccharide, or a synthetic polymer composed of a plurality of repeating units.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

### Dosage

Embodiments of the disclosure relate to a dosage form comprising one or more compounds of the disclosure that can provide peak plasma concentrations of the compound of between about 0.001 to 2 mg/ml, 0001 to 1 mg/ml, 0.0002 to 2 mg/ml, 0.005 to 2 mg/ml, 001 to 2 mg/ml, 0.05 to 2 mg/ml, 0.001 to 0.5 mg/ml, 0.002 to 1 mg/ml, 0.005 to 1 mg/ml, 0.01 to 1 mg/ml, 005 to 1 mg/ml, or 0.1 to 1 mg/ml. The disclosure also provides a formulation or dosage form comprising one or more compound of the disclosure that provides an elimination t½ of 0.5 to 20 h, 0.5 to 15 h, 0.5 to 10 h, 0.5 to 6 h, 1 to 20 h, 1 to 15 h, 1 to 10 h, or 1 to 6 h.

A subject may be treated with a compound of the disclosure or composition or unit dosage thereof on substantially any desired schedule. They may be administered one or more times per day, in particular 1 or 2 times per day, once per week, once a month or continuously. However, a subject may be treated less frequently, such as every other day or once a week, or more frequently. A compound or composition may be administered to a subject for about or at least about 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously.

A beneficial pharmacokinetic profile can be obtained by the administration of a formulation or dosage form suitable for once, twice, or three times a day administration, preferably twice a day administration comprising one or more compound of the disclosure present in an amount sufficient to provide the requited dose of the compound. The required dose of a compound of the disclosure administered once twice, three times or more daily is about 0.01 to 3000 mg/kg, 0.01 to 2000 mg/kg, 0.5 to 2000 mg/kg, about 0.5 to 1000 mg/kg, 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 50 mg/kg, 0.1 to 20 mg/kg, 0.1 to 10 mg/kg, 0.1 to 6 mg/kg, 0.1 to 5 mg/kg, 0.1 to 3 mg/kg, 0.1 to 2 mg/kg, 0.1 to 1 mg/kg, 1 to 1000 mg/kg, 1 to 500 mg/kg, 1 to 400 mg/kg, 1 to 300 mg/kg, 1 to 200 mg/kg, 1 to 100 mg/kg, 1 to 50 mg/kg, 1 to 20 mg/kg, 1 to 10 mg/kg, 1 to 6 mg/kg, 1 to 5 mg/kg, or 1 to 3 mg/kg, or 1 to 2.5 mg/kg, or less than or about 10 mg/kg, 5 mg/kg, 2.5 mg/kg, 1 mg/kg, or 0.5 mg/kg twice daily or less

Certain dosage forms and formulations may minimize the variation between peak and trough plasma and/or brain levels of compounds of the disclosure and in particular provide a sustained therapeutically effective amount of the compounds.

A medicament or treatment of the disclosure may comprise a unit dosage of at least one compound of the disclosure to provide therapeutic effects. A “unit dosage” or “dosage unit” refers to a unitary, i.e. a single dose, which is capable of being administered to a patient, and which may be readily handled and packed, remaining as a physically and chemically stable unit dose comprising either the active agents as such or a mixture with one or more solid or liquid pharmaceutical excipients, carriers, or vehicles.

The term “unit dosage form” as used herein refers to physically discrete units suitable as unitary dosages for human patients and other mammals with each unit containing a predetermined quantity of active material calculated to produce the desired therapeutic effect in association with suitable pharmaceutical carriers or excipients. The compositions according to the present disclosure may be formulated in a unit dosage form. A single daily unit dose also may be divided into 2 or 3 unit doses that are taken at different times throughout the day, or as a controlled release form, so as to reduce adverse side-effects as much as possible.

The term “dosage form” as used herein refers to a composition or device comprising a compound of the disclosure and optionally pharmaceutically acceptable carrier(s), excipient(s), or vehicles. A dosage form may be an immediate release dosage form or a sustained release, dosage form. An “immediate release dosage form” refers to a dosage form which does not include a component for sustained release i.e., a component for slowing disintegration or dissolution of an active compound. These dosage forms generally rely on the composition of the drug matrix to effect the rapid release of the active ingredient agent. By “sustained release dosage form” is meant a dosage form that releases active compound for many hours. In an aspect, a sustained dosage form includes a component for slowing disintegration or dissolution of the active compound. A dosage form may be a sustained release formulation, engineered with or without an initial delay period. Sustained release dosage forms may continuously release drug for sustained periods of at least about 4 hours or more, about 6 hours or more, about 8 hours or more, about 12 hours or more, about 15 hours or more, or about 20 hours to 24 hours. A sustained release dosage form can be formulated into a variety of forms, including tablets, lozenges, gelcaps, buccal patches, suspensions, solutions, gels, etc. In aspects of the disclosure the sustained release form results in administration of a minimum number of daily doses.

### Discussion

In accordance with the purpose(s) of the present disclosure, as embodied and broadly described herein, embodiments of the present disclosure, in some aspects, relate to methods of reducing inflammation, methods of reducing cognitive decline, and pharmaceutical compositions for reducing cognitive decline.

The present disclosure includes a method for reducing cognitive decline in a subject. Advantageously, the treatments described herein can be administered peripherally and do not require brain-penetrating agents.

Embodiments of the present disclosure include a method for reducing inflammation in a subject, in which an EP2 signal is inhibited.

Embodiments of the present disclosure include a method for reducing cognitive decline in a subject, comprising inhibiting an EP2 signal by peripherally administering a composition comprising an EP2 antagonist to a subject in need thereof.

Embodiments of the present disclosure include a pharmaceutical composition comprising an EP2 antagonist.

Aging is characterized by the development of persistent pro-inflammatory responses that promote diseases like atherosclerosis, metabolic syndrome, cancer, and frailty. The aging brain is vulnerable to inflammation, as demonstrated by the high prevalence of age-associated cognitive decline and Alzheimer's dementia. Systemically, circulating pro-inflammatory factors can promote cognitive decline and in brain, microglia lose the ability to maintain immune homeostasis and clear misfolded proteins that are associated with neurodegeneration. However, the underlying mechanisms that initiate and sustain maladaptive inflammation with aging are not well defined. The present disclosure shows that in aging mice, myeloid cell bioenergetics are suppressed in response to increased signaling by the lipid messenger prostaglandin E2 (PGE2), a major modulator of inflammation. In aging macrophages and microglia, PGE2 signaling through its EP2 receptor promotes the sequestration of glucose into glycogen, reducing glucose flux and mitochondrial respiration. This energy deficient state shifts myeloid polarization state and immune responses towards a maladaptive pro-inflammatory phenotype and is further aggravated by dependence of aged myeloid cells on glucose as a principal fuel source. In aged mice, inhibition of myeloid EP2 signaling restores youthful energy metabolism in peripheral macrophages and microglia, rejuvenates systemic and brain inflammatory states, and prevents loss of hippocampal synaptic plasticity and spatial memory. Moreover, blockade of peripheral myeloid EP2 signaling is sufficient to restore cognition in aged mice. The studies in the present example suggest that cognitive aging may not be a static or irrevocable condition but can be reversed by reprogramming myeloid glucose metabolism to restore youthful immune function.

A hallmark of aging is the appearance of sustained pro-inflammatory responses and reduced clearance of pathogenic materials. Systemically, aging is accompanied by a skewing of the immune system towards the myeloid cell lineage and an increase in circulating pro-inflammatory factors. In the aging brain, functional degradation of microglia leads to the accumulation of neurotoxic misfolded proteins, a loss of trophic factors that support neurons, and a failure to maintain a homeostatic microenvironment. Brain and systemic myeloid responses are tightly linked to the development of age-associated cognitive decline and Alzheimer's disease, where human genetics confirm a role for myeloid responses in increasing disease risk. The underlying mechanisms responsible for the development of maladaptive myeloid phenotypes in aging are not well understood, however recent studies point to an important role for cellular energy metabolism in regulating immune activation state and function. To maintain homeostasis, immune cells require robust glycolytic and mitochondrial metabolism to meet demand for energy and biosynthetic precursors. Indeed, recent studies indicate that aging macrophages display profound decreases in glycolysis and mitochondrial oxidative phosphorylation that lead to dysregulated immune responses.

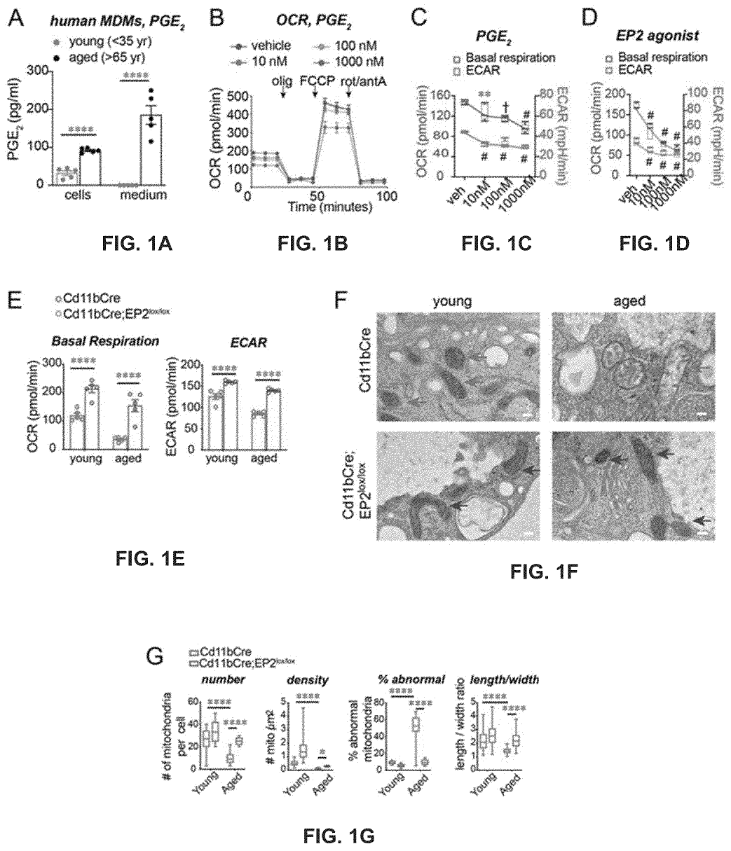

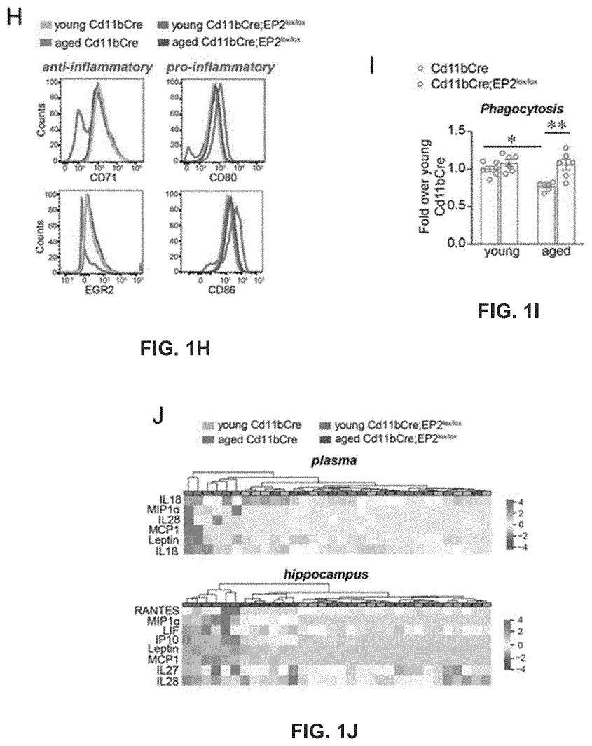

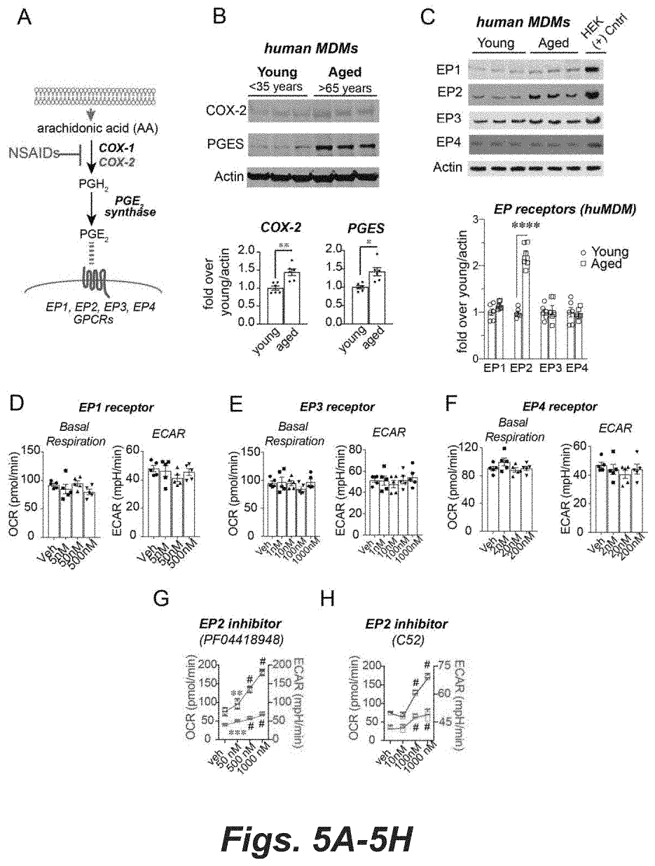

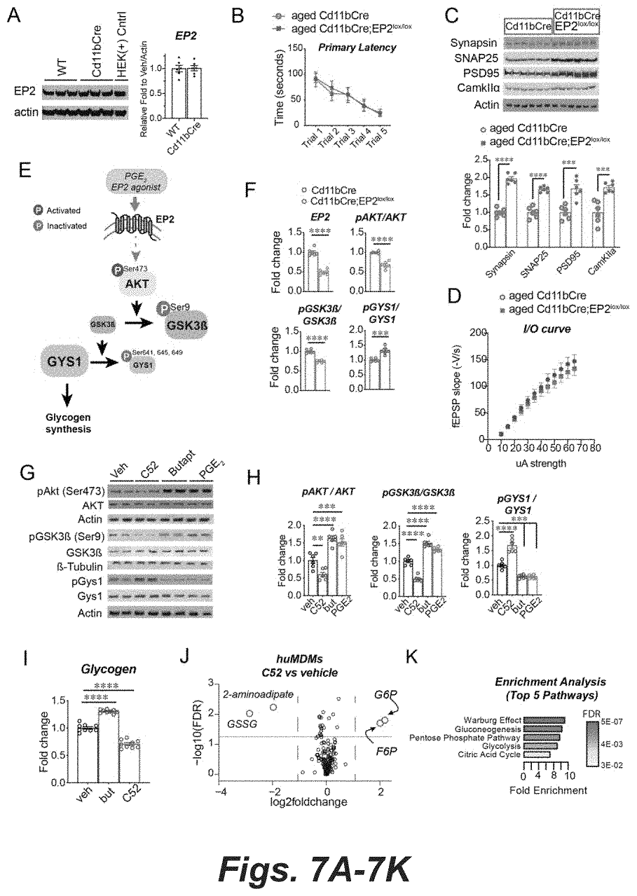

The lipid messenger prostaglandin E2 (PGE2) is a downstream product of the cyclooxygenase-2 (COX-2) pathway (FIG. 7A) and is a major modulator of inflammation. PGE2 levels increase in aging and in neurodegenerative disease. It was hypothesized that age-related increases in PGE2 may connect development of maladaptive inflammation to cognitive decline in aging mice. An age-associated increase in the synthesis of PGE2 in human monocyte-derived macrophages (human MDMs) was identified from subjects older than 65 years of age (FIG. 1A and FIG. 7B). Given the link between cellular metabolism and myeloid cell function, it was first determined whether PGE2 signaling affected macrophage energy metabolism. Simultaneous measurement of mitochondrial respiration and glycolysis was determined in human MDMs by measuring the influx of oxygen for mitochondrial respiration and proton efflux from glycolysis-derived lactate. Dose-dependent stimulation with PGE2 for 20 hours decreased glycolysis (extracellular acidification rate or ECAR) and suppressed oxygen consumption (OCR) (FIG. 1B-1C). Although PGE2 signals through four G-protein coupled receptors, EP1-4, the suppressive effect of PGE2 was mediated by the EP2 receptor which was expressed at highest levels in aged human MDMs (FIG. 5C-F). In contrast to PGE2 and the EP2 selective ligand butaprost, the selective EP2 inhibitors PF04418948 and compound 52 (C52) increased macrophage basal OCR and ECAR (FIG. 5G-H). These data suggest that inhibition of PGE2 EP2 signaling might enhance energy production in aging myeloid cells.

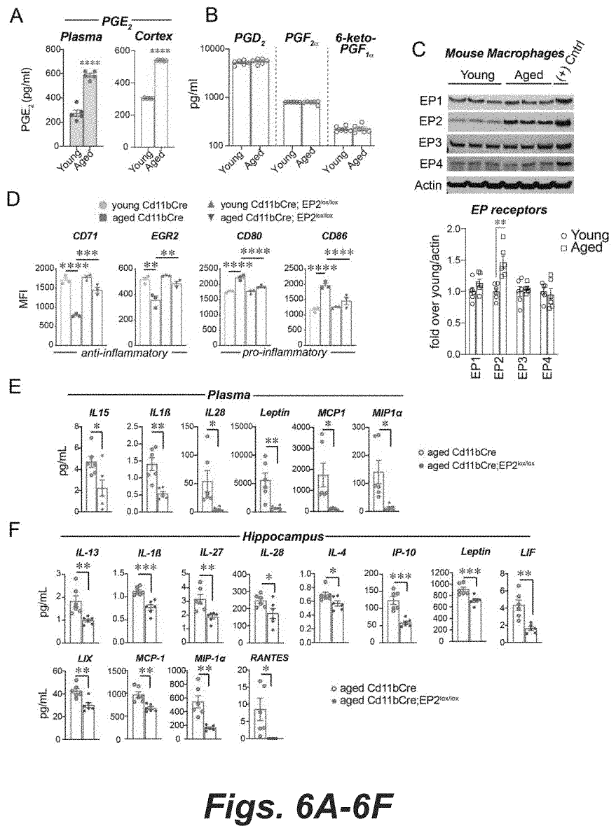

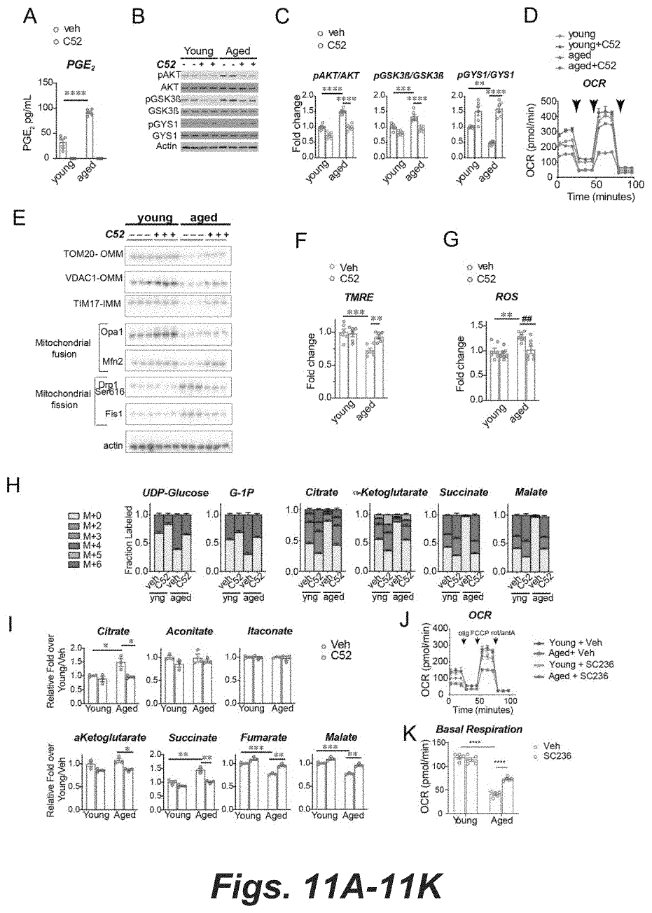

Also confirmed were significant increases in PGE2 levels in 20 month old mice both in plasma and cerebral cortex and a specific increase in myeloid EP2 receptor levels (FIG. 6A-6C). Accordingly, effects of myeloid cell-specific deletion of EP2 in aging Cd11bCre;EP2^{lox/lox} mice were examined, where levels of EP2 are decreased by 50% in myeloid lineage cells. OCR was suppressed in peritoneal macrophages isolated from aged Cd11bCre mice as compared to young mice, but in macrophages derived from aged Cd11bCre;EP2^{lox/lox} mice, OCR and ECAR were restored to young levels (FIG. 1E). Moreover, transmission electron microscopy (TEM) revealed significant abnormalities in mitochondrial morphology, numbers, and density in peritoneal macrophages derived from aged Cd11bCre mice that were absent in macrophages isolated from aged Cd11bCre;EP2^{lox/lox} mice (FIG. 1F-1G). Macrophages isolated from aged Cd11bCre mice exhibited a pro-inflammatory phenotype as compared to young macrophages, however the polarization state of aged Cd11bCre;EP2^{lox/lox} macrophages was indistinguishable from that of young macrophages of either genotype (FIG. 1H and FIG. 6D). In addition, myeloid EP2 knockdown in aged mice restored phagocytic ability of macrophages to youthful levels (FIG. 11). Multiplex profiling of immune factors in aged Cd11bCre;EP2^{lox/lox} mice demonstrated a distinctive pattern of pro-inflammatory factor enrichment in aged Cd11bCre mice, whereas Cd11bCre;EP2^{lox/lox} aged mice showed profiles more similar to young mice (FIG. 1J; FIG. 6E-6F). This youthful immune profile was observed both in plasma and in hippocampus, the latter likely reflecting dual effects of systemic myeloid as well as brain microglial EP2 inhibition. Thus, myeloid knockdown of EP2 receptor in aging mice restored cell bioenergetics, polarization state, and phagocytic capability to youthful levels.

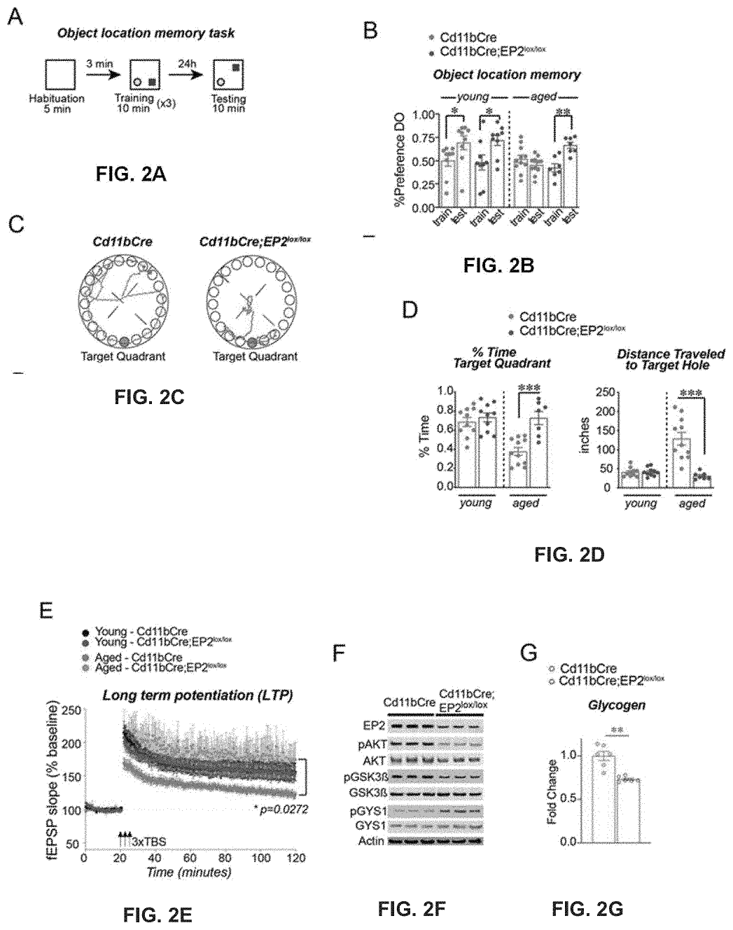

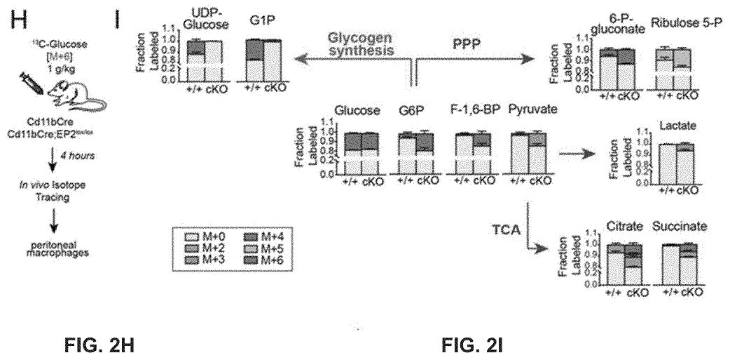

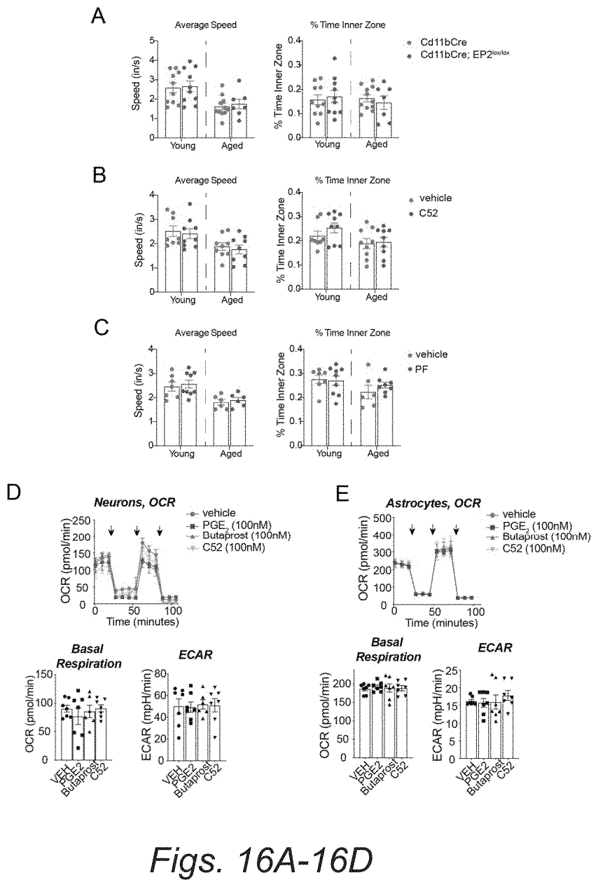

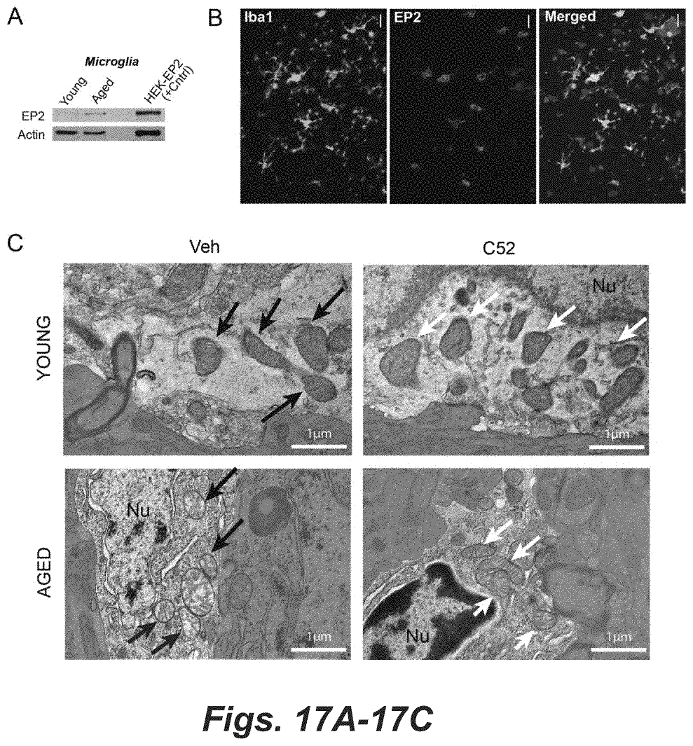

Given the association between inflammation and cognitive impairment, it was reasoned that a reduction in myeloid EP2 signaling may improve cognitive function in aging mice. Hippocampal-dependent spatial memory is particularly vulnerable to aging, so the performance was tested in the object location memory task and the Barnes maze task. In both tasks, the performance of aged Cd11bCre;EP2^{lox/lox} mice was indistinguishable from that of young mice of either genotype, in sharp contrast to aged Cd11bCre control mice (FIG. 2A-D; FIGS. 7A-7B and 16A). Consistent with this behavioral rescue, levels of hippocampal pre- and post-synaptic proteins, which decrease in aging, were increased in aged Cd11bCre;EP2^{lox/lox} hippocampi (FIG. 7C). Measurement of hippocampal synaptic plasticity in aged Cd11bCre;EP2^{lox/lox} mice, assayed by electrophysiological recordings of the CA3 to CA1 Shaffer collateral pathway, demonstrated a robust improvement in long-term potentiation, a cellular correlate of learning and memory that deteriorates with aging (FIG. 2E; FIG. 7D). Thus, reduction of myeloid EP2 signaling in aging mice restored cellular energy metabolism, systemic and brain inflammation, and hippocampal plasticity and memory function to youthful levels.

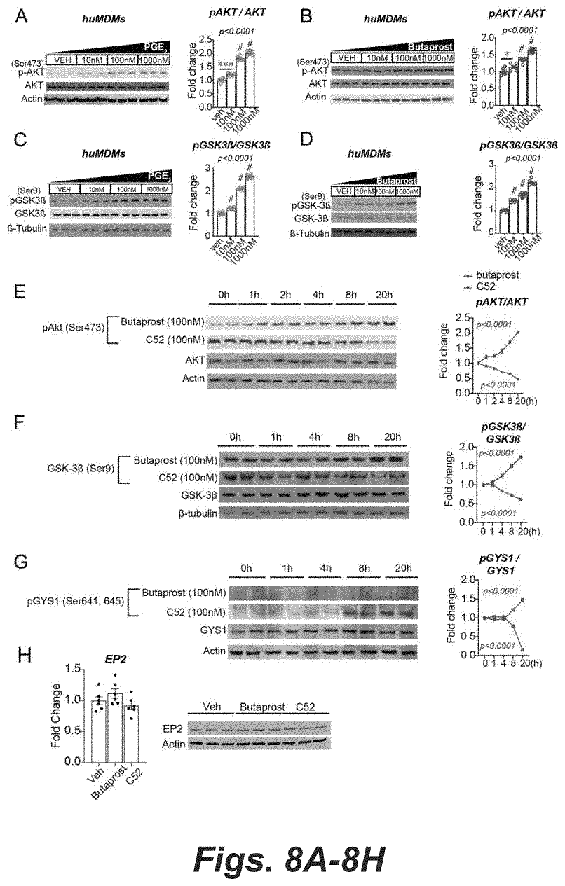

To understand how inhibition of EP2 signaling elicited such beneficial effects in the context of aging, signaling cascades downstream of EP2 (FIG. 7E) were examined. PGE2 EP2 signaling activated protein kinase B (AKT) which then phosphorylated and inactivated GSK3β at Ser9, thereby permitting activity of glycogen synthase (GYS1), the rate limiting enzyme in glycogen synthesis. Reduction of EP2 signaling in 6 mo Cd11bCre;EP2^{lox/lox} macrophages or in wild-type peritoneal macrophages exposed to EP2 inhibitor led to GSK311-mediated inactivation of GYS1 and a reduction of intracellular glycogen levels (FIG. 2F-2G; FIG. 7F). Additional pharmacologic validation in human MDMs confirmed a significant suppression of glycogen synthesis with EP2 inhibition (FIGS. 7G-7I and 8A-8H). In addition, untargeted metabolomics in human MDMs treated with EP2 inhibitor revealed increased glucose-6P and fructose-6P and decreased oxidized glutathione (GSSG) (FIG. 7J-7K), suggesting a suppressive effect of EP2 signaling on glucose flux down the glycolytic pathway.

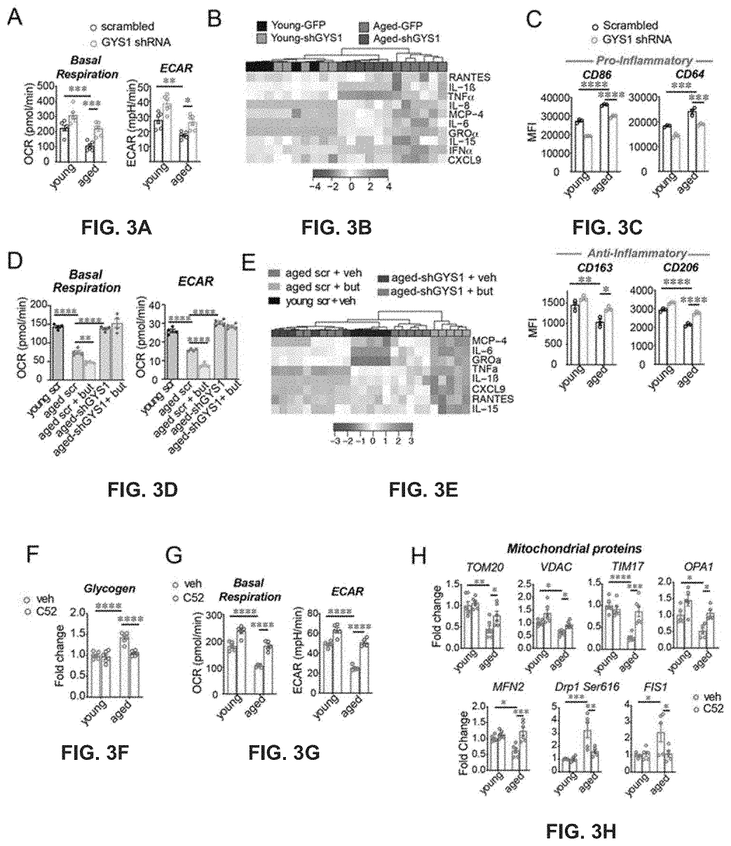

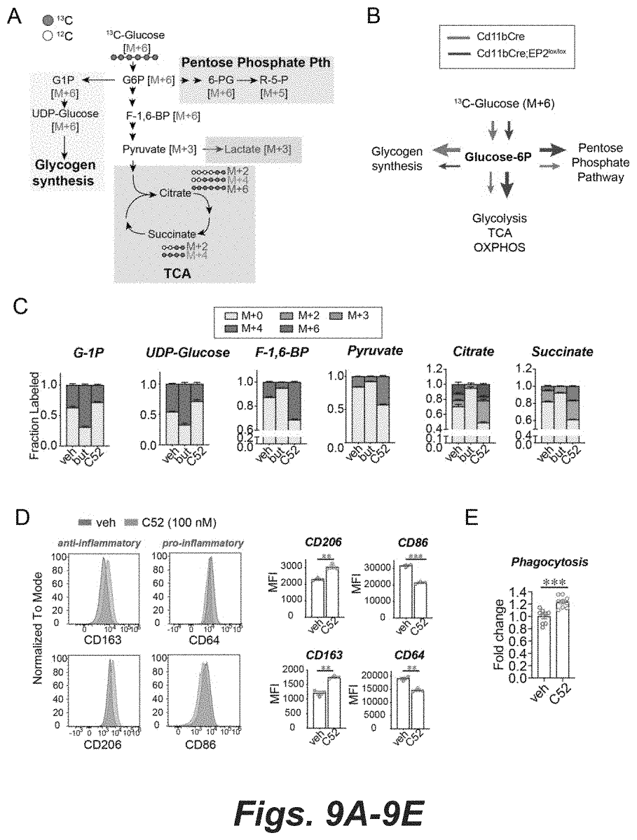

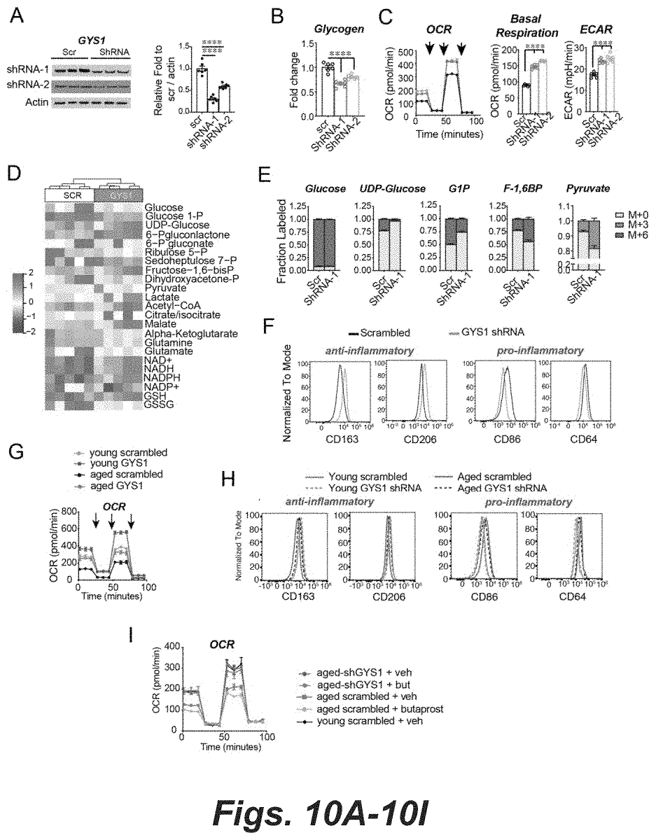

To further validate the metabolic impact of EP2 signaling in macrophages, [U-¹³C] glucose was administered to aged Cd11bCre and Cd11bCre;EP2^{lox/lox} mice, isolated macrophages four hours later, and measured labeling in glycolytic and TCA metabolites (FIG. 2H-2I). Loss of macrophage EP2 resulted in lower in vivo incorporation of ¹³C-glucose into UDP-glucose, the precursor of glycogen, and more incorporation into glycolytic and citric acid cycle intermediates (FIG. 2I and FIG. 9A-9B), consistent with increased glucose flux. ¹³C-glucose labeling was also carried out in human MDMs treated with EP2 inhibitor C52, where incorporation of label similarly decreased in glycogen precursors and increased in glycolytic and TCA intermediates; the converse occurred following EP2 agonism with butaprost (FIG. 9C). Recent studies demonstrate that cellular bioenergetics regulate macrophage polarization state via the accumulation of TCA cycle intermediates such as pro-inflammatory succinate. Consistent with this, the metabolic consequences of EP2 inhibition in human MDMs included a shift in polarization to a more anti-inflammatory activation state and more robust phagocytic capacity (FIG. 9D-9E). These genetic and pharmacologic findings link EP2-driven changes in glycogen synthesis and glucose flux to macrophage cell polarization and phagocytic capability. Next the effect of lowering glycogen synthesis was directly tested on macrophage energy metabolism, cytokine production, and polarization state. Knockdown of rate-limiting GYS1 in human MDMs decreased glycogen levels and increased basal respiration and ECAR (FIG. 10A-10C). Consistent with this, targeted metabolomics of human MDMs depleted of GYS1 showed improved bioenergetics, with increases in glycolytic intermediates, NAD+ and NADH, NADPH, and reduced glutathione (FIG. 10D). A shift away from glycogen synthesis and towards glycolysis was further confirmed using ¹³C-glucose isotope tracing in these cells (FIG. 10E). Consistent with the regulation of immune polarization state by metabolic state, GYS1 deficiency promoted a more anti-inflammatory activation state in human MDMs (FIG. 10F). Whether GYS1 deficiency in aged macrophages could restore youthful mitochondrial function and polarization state was then tested. Indeed, knockdown of GYS1 in human MDMs derived from aged subjects (>65 years of age) restored basal respiration and ECAR to levels observed in human MDMs derived from young subjects (<35 years of age; FIG. 3A and FIG. 10G). Knockdown of GYS1 in aged human MDMs restored pro-inflammatory cytokine generation and polarization state to youthful levels (FIG. 3B-3C; FIG. 10H). Moreover, activation of EP2 signaling with butaprost did not alter the bioenergetic or immune factor phenotypes of aged human MDMs lacking GYS1 (FIGS. 3D-3E and FIG. 10I), confirming GYS1 as a critical effector of EP2-mediated effects on myeloid metabolism and immune state.

The effect of pharmacologic inhibition of inflammatory EP2 in aged human MDMs was then tested. PGE2 levels, EP2 receptor expression, and downstream phosphorylation of AKT/GSK3β and activation of GYS1 were significantly higher in human MDMs derived from aged as compared to young subjects (FIG. 1A, FIGS. 5C, and 11A-11C). Consistent with previous genetic data in aged Cd11bCre;EP2^{lox/lox} mice, pharmacologic inhibition of EP2 in aged human MDMs normalized glycogen levels and restored OCR and ECAR to youthful levels (FIG. 3F-G; FIGS. 11D, and 11J-11K). EP2 blockade in aged huMDMs restored protein levels, mitochondrial membrane potential, and reactive oxygen species (ROS) to those of young human MDMs (FIG. 3H and FIGS. 11E-11G). Targeted metabolomics and ¹³C-glucose isotope tracing confirmed the rescue of glycolysis and TCA intermediates in aged human MDMs with inhibition of EP2 which demonstrated improved phagocytosis (FIG. 3I-3J and FIGS. 11H-11I).

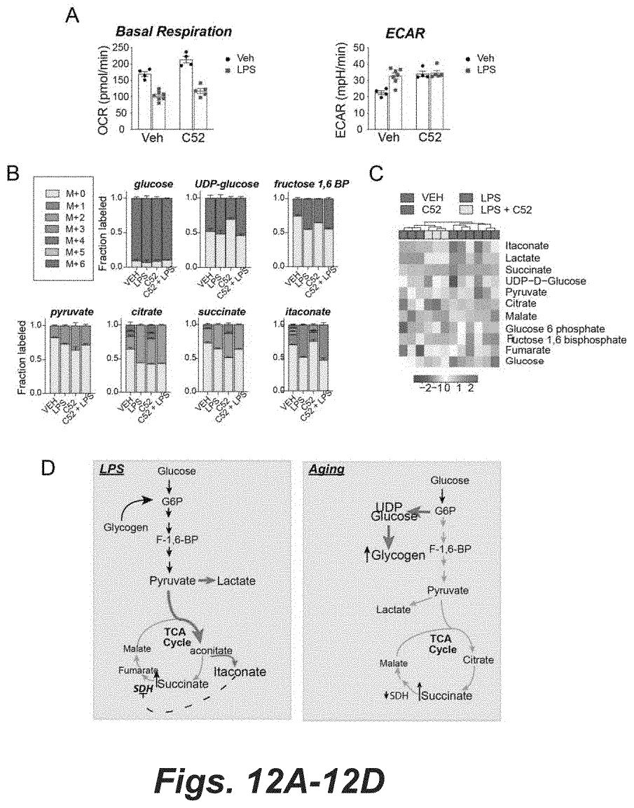

Previous work has demonstrated that the activity of Complex II of the electron transport chain (succinate dehydrogenase or SDH) is suppressed in aged macrophages. Low SDH activity leads to accumulation of the TCA metabolite succinate which stabilizes activity of Hif-1□, an activator of pro-inflammatory cytokine expression, leading to pro-inflammatory polarization. Accumulation of succinate is also observed in macrophages acutely stimulated with lipopolysaccharide (LPS); in this context, SDH activity is suppressed by itaconate that is generated from increased metabolism of aconitate by Irg1. However, EP2 blockade did not overcome LPS-mediated changes in OCR and ECAR or succinate accumulation (FIG. 12A-C). Together with the observation that aconitate and itaconate levels do not change in aged macrophages, these data suggest that the metabolic state of aged macrophages is distinct from that of LPS-activated macrophages (FIGS. 11I and 12D).

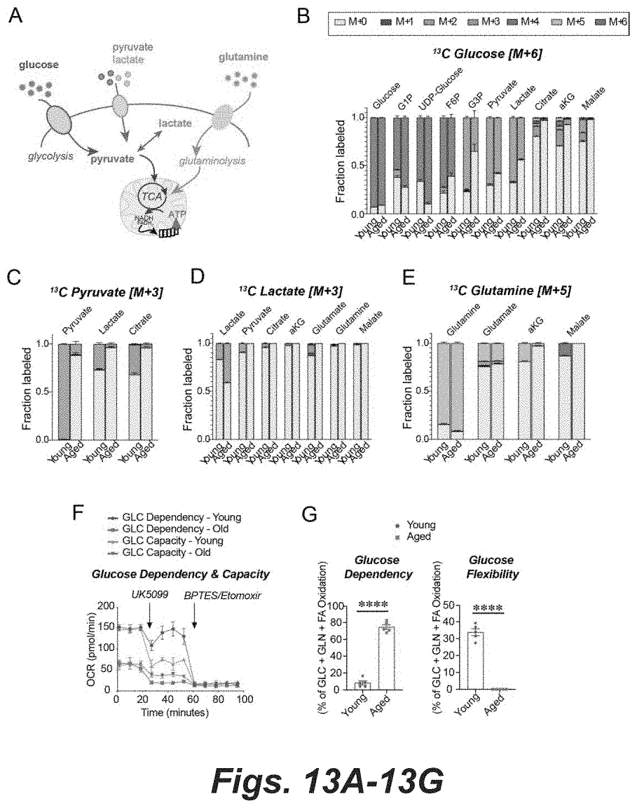

Glycogen is a fuel source that is used by many cell types, including immune cells. However, in aging macrophages, the reverse situation develops wherein glucose is sequestered into glycogen from increased EP2-driven GYS1 activity, leading to bioenergetic insufficiency from lower glucose flux to mitochondria. As cells are normally capable of utilizing additional fuel sources, for example glutamine or lactate, it was determined whether this metabolic vulnerability resulted from an inability of aged macrophages to metabolize other fuel substrates (FIG. 13A). Incubation of young and aged human MDMs with ¹³C-isotopes of glutamine, pyruvate, lactate, and glucose (FIGS. 13B-13E) revealed a fundamental dependence of aged human MDMs on glucose as a fuel source. Whereas isotope tracing in young macrophages revealed stable incorporation of ¹³C derived from glucose, pyruvate, lactate, and glutamine into glycolytic and TCA intermediates, aged macrophages metabolized only glucose. Fuel flexibility of young versus aged human MDMs was assayed by independently inhibiting fatty acid oxidation, glutamine metabolism, and pyruvate transport into the mitochondria (FIGS. 13F-13G). Consistent with the isotope tracing, aged macrophages demonstrated significant glucose dependence when pyruvate transport was inhibited, indicating that glucose availability and flux are essential for oxidative phosphorylation in aged macrophages.

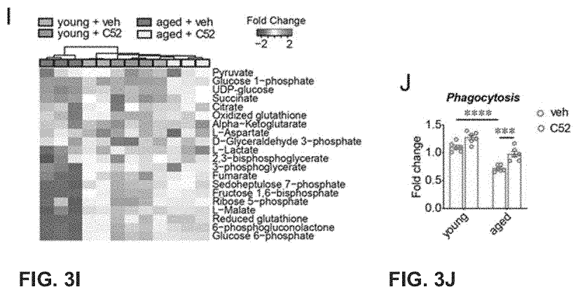

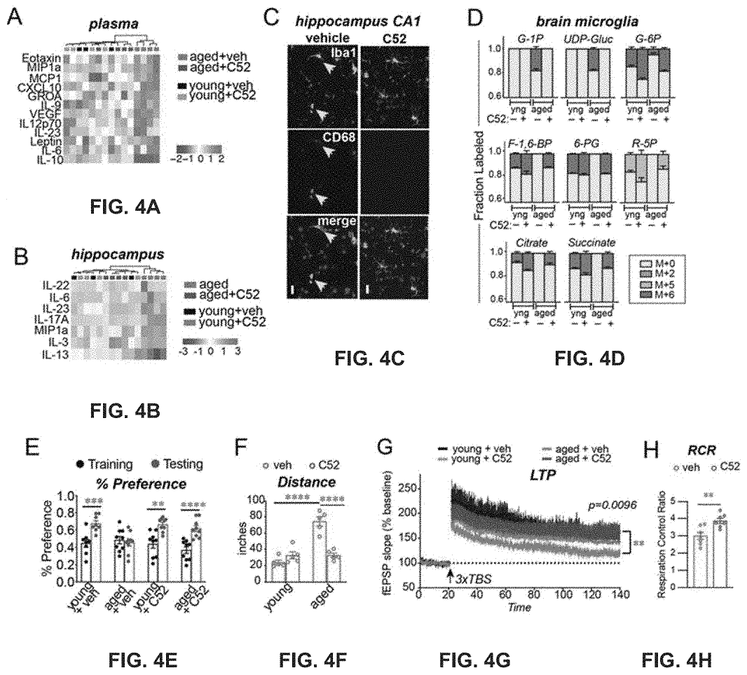

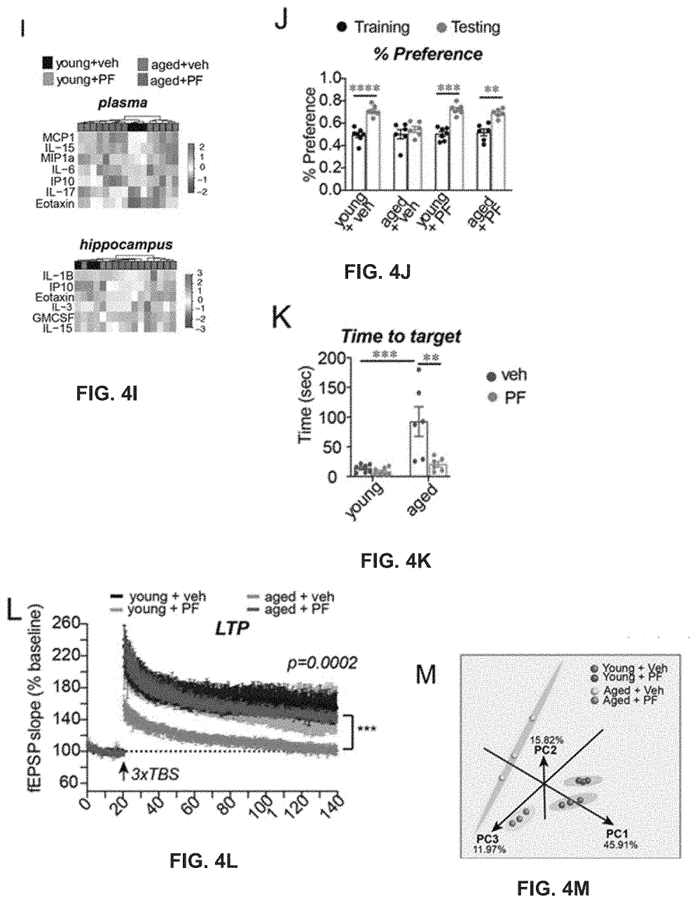

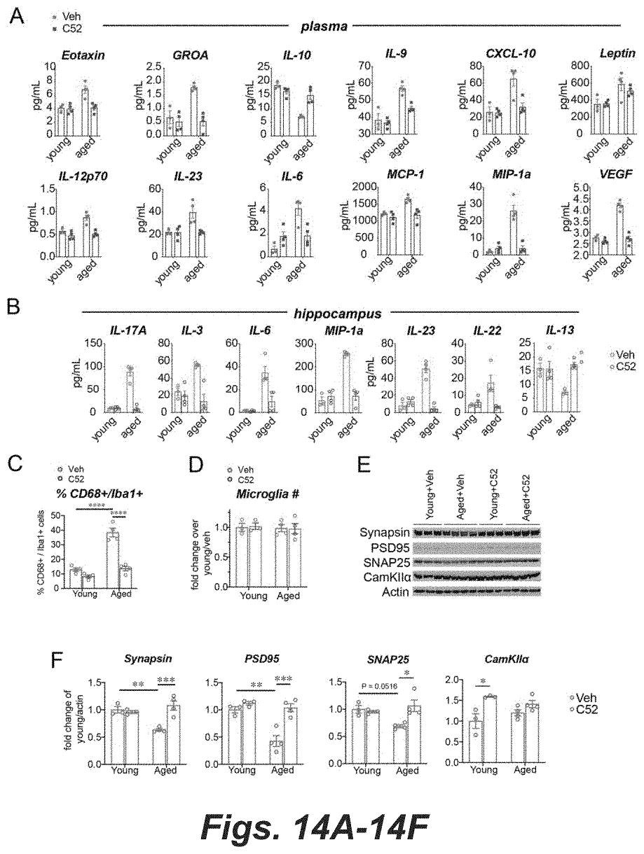

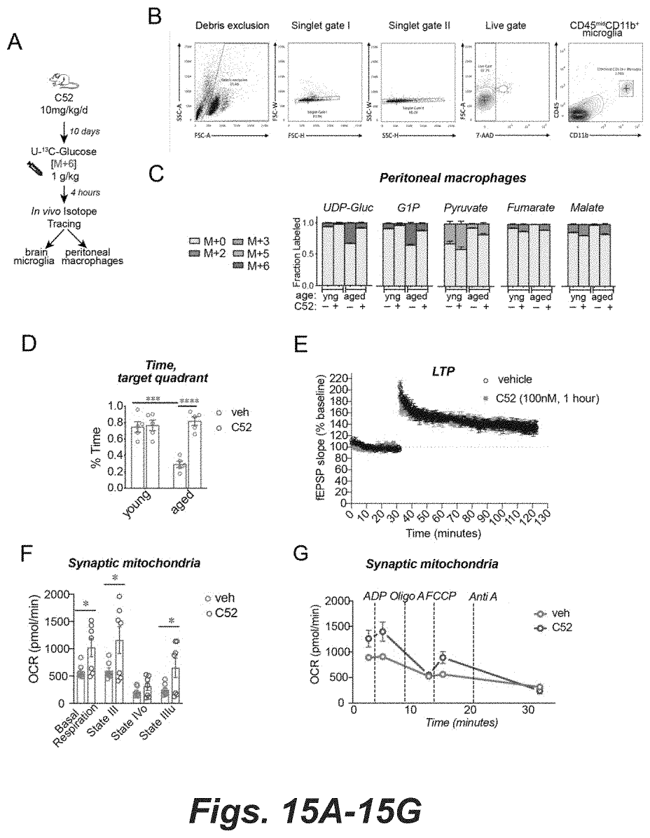

Whether in vivo pharmacologic inhibition of EP2 signaling in aged mice might elicit effects on inflammation and cognitive function similar to those observed in aged Cd11bCre;EP2^{lox/lox} mice was then tested. Administration of the brain-penetrant EP2 inhibitor Compound 52 for 1 month restored pro- and anti-inflammatory factors in plasma and in hippocampus to youthful levels (FIGS. 4A-B; FIGS. 14A-14B) and reduced levels of CD68, a marker of inflammatory microglial activation in aged mice (FIG. 4C, FIGS. 14C-14D, and 14A-14B). Brain microglia and peritoneal macrophages were examined for in vivo isotope labeling of their metabolic intermediates from ¹³C-glucose isotope administered 4 hours prior (FIG. 4D and FIGS. 15A-15C). Isotope labeling of brain microglia and peritoneal macrophages revealed a similar pattern of reduced glycogen synthesis and enhanced glycolytic and TCA cycle labeling with EP2 inhibition as seen previously in vitro FIGS. 9B-9C) and in vivo in aged Cd11bCre;EP2^{lox/lox} mice (FIG. 2I). Further validation of microglial bioenergetic rescue was performed using TEM of hippocampus (FIG. 14C) where morphologic features of aged microglial mitochondria in mice treated with C52, including cristae formations and electron density, resembled those of young microglial mitochondria. Functionally, EP2 blockade led to resolution of age-associated spatial memory deficits in both the Novel Object Location and Barnes Maze tasks (FIG. 4E-4F; FIGS. 15D and 16B) and normalization of synaptic proteins to youthful levels (FIG. 14E-F), consistent with previous findings in aged Cd11bCre;EP2^{lox/lox} mice. Electrophysiological recordings to assess hippocampal long-term plasticity in aged mice showed a restoration of youthful LTP with EP2 inhibition (FIG. 4G; FIGS. 15E and 16D-16E), similar to observations in aged Cd11bCre;EP2^{lox/lox} mice (FIG. 2E).

Healthy mitochondria are critical for synaptic neurotransmission and plasticity. The integrity of synaptic mitochondria was assessed by determining the extent of coupling between the electron transport chain (ETC) and oxidative phosphorylation of ADP to ATP in synaptosomes (FIG. 4H and FIG. 15F-G). Basal respiration (State II) and ADP-supplemented respiration (state III) reflect both electron transport and ATP generation and increased by two-fold with EP2 blockade. Maximal respiration (State IIIu) following application of the H+ gradient uncoupler FCCP was also higher with EP2 inhibition, and State IVo, reflecting blockade of ATP synthase with oligomycin was unchanged. The respiratory control ratio (RCR), represented as the ratio of State III/State IVo, was significantly increased following EP2 inhibition (FIG. 4H), indicating improved mitochondrial coupling of electron transport and ATP synthesis. These data demonstrate that inhibition of inflammatory EP2 improves mitochondrial health of aged synapses.

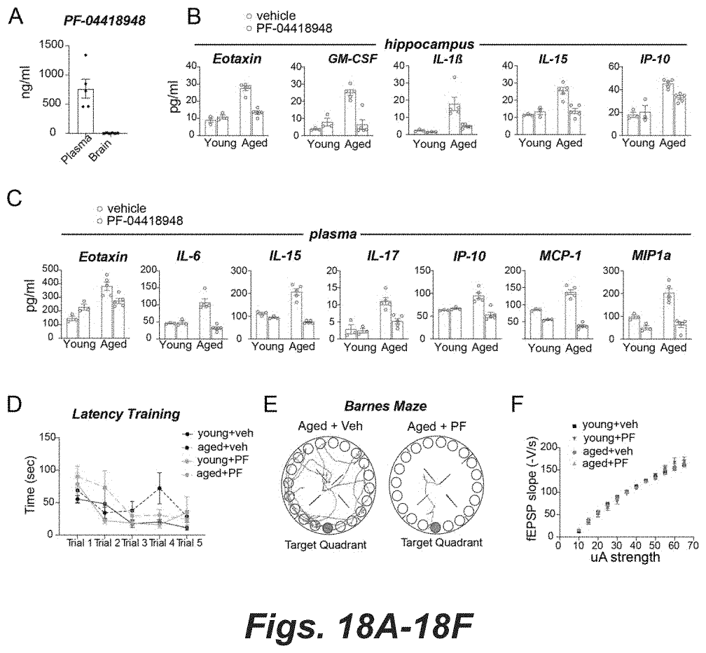

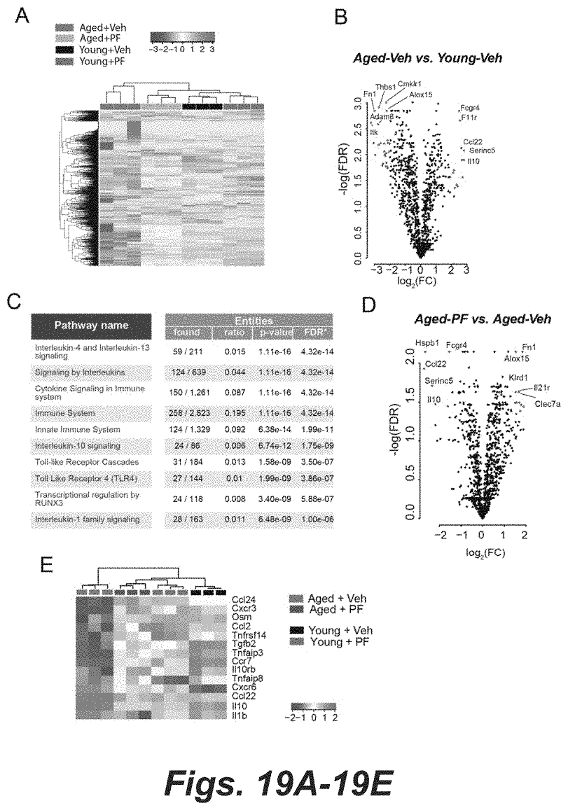

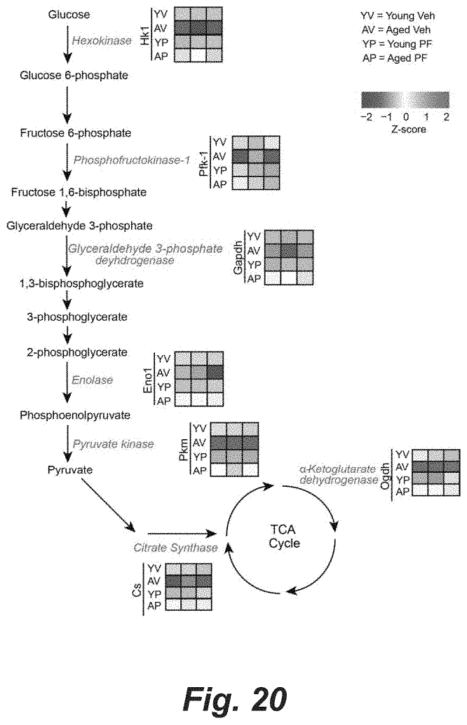

Since the EP2 receptor is expressed in brain microglia as well as peripheral myeloid cells, whether peripheral EP2 blockade would be sufficient to reverse age-associated inflammation and hippocampal memory deficits was examined. Accordingly, the effects of the selective brain-impermeant EP2 antagonist PF-04418948 were tested in aging mice (FIGS. 18A and 5G). Peripheral inhibition of EP2 signaling for 6 weeks reduced levels of pro-inflammatory factors not only in blood but also in hippocampus (FIG. 4I and FIG. 18B-C). Remarkably, peripheral EP2 blockade restored hippocampal memory function as well as LTP to youthful levels (FIG. 4K-L and FIGS. 16C and 18D-18F). Transcriptomic analysis of macrophages derived from aged, EP2-inhibited mice revealed a significant shift away from vehicle-treated aged mice toward young macrophages (FIG. 4M and FIG. 19A). Analysis of aged macrophages revealed upregulation of pro-inflammatory gene transcripts and downregulation of glycolytic and TCA gene transcripts in aged macrophages that were reciprocally reversed with PF-04418948 administration (FIGS. 19B-19E and 20A).

The development of maladaptive inflammation and cognitive decline in aging may not be a static or permanent condition, but rather can be reversed by inhibiting inflammatory PGE2 signaling through the myeloid EP2 receptor. Aging is associated with a significant increase in pro-inflammatory PGE2 signaling in myeloid cells that drives sequestration of glucose into glycogen through the AKT/GSK3β/GYS1 pathway and away from generation of ATP. Secondly, a fundamental vulnerability of aging myeloid cells in which they become dependent on glucose and unable to utilize alternate energy sources to support mitochondrial respiration was found. These two mechanisms converge, leading to depletion of glucose flux to the TCA and development of an energy deficient state that drives pro-inflammatory and maladaptive immune responses. Myeloid metabolism regulates phagocytosis and macrophage polarization state via accumulation of TCA cycle intermediates like succinate. Third, by directing glucose towards ATP production, as opposed to glycogen storage in aging myeloid cells, inhibition of myeloid EP2 signaling, either genetically or pharmacologically, reverts polarization states to more homeostatic and youthful anti-inflammatory states that prevent age-associated cognitive decline. Finally, peripheral EP2 blockade is sufficient to re-establish youthful immune homeostasis not just in the blood, but in the brain, and to restore hippocampal function and plasticity in aged mice. The present study suggests that the myeloid EP2 signaling cascade may drive a strong component of aging. These findings are also consistent with a feed-forward loop involving the inflammatory cyclooxygenase-2/PGE2/EP2 cascade, wherein increasing PGE2 signaling via the EP2 receptor induces additional COX-2 expression and activity, further amplifying downstream PGE2 generation and signaling. Thus, inhibition of EP2-dependent changes in myeloid metabolism may represent a new approach to disorders of aging, with greater specificity than the use of non-steroidal anti-inflammatory drugs that target COX-2 and COX-1 and suppress both beneficial and toxic prostaglandin signaling pathways.

### Methods

Animals—This study was conducted in accordance with National Institutes of Health (NIH) guidelines and the Institutional Animal Care and Use Committee at Stanford University approved protocols. All mice were housed in an environmentally controlled, pathogen-free barrier facility on a 12 h light-dark cycle, temperature, and humidity, with food and water available ad libitum. C57BL/6J mice were bred using mice purchased from Jackson laboratories or obtained from the NIH aged rodent colony. Young (2-3 mo) and aged (22-24 mo) C57BL/6J mice used in each experiment were aged- and source-matched. Cd11bCre and Cd11bCre;EP2^{lox/lox} mice have been previously described.

### Materials

PGE₂, Butaprost, PF04418948 and Iloprost were purchased from Millipore-Sigma (Burlington, Mass., USA). ONO-AE248 and ONO-AE1-329 were gifts from Ono Pharmaceuticals (Osaka, Japan). Compound 52 (Charnwood Molecular Ltd, Loughborough, UK) and PF04418948 (Millipore-Sigma, Burlington, Mass., USA) were resuspended in 40% PEG (Sigma-Aldrich) and 60% of a 30% Kolliphor HS15 solution (Sigma-Aldrich), and administered orally at 10 mg/kg/d and 2.5 mg/kg/d, respectively. U-¹³C-Glucose, U-¹³C-Lactate, U-¹³C-Glutamine, and U-¹³C-Pyruvate were purchased from Cambridge Isotopes (Tewksbury, Mass., USA). HUSH-29 plasmids containing shRNAs to human GYS1 were purchased from Origene Technologies (Rockville, Md., USA). Human monocyte derived macrophages were incubated with 1640 Media with GlutaMAX+HEPES without sodium pyruvate (ThermoFisher, catolog No. 72400146). Mouse macrophages were incubated in DMEM without sodium pyruvate (Sigma-Aldrich, Catalog No. D5796).

### Real-Time Oxygen Consumption Rate (OCR) and ECAR

Cells were plated at 1.8×10⁶ cells per well in a Seahorse XF24 Cell Culture Microplate (Agilent). Cells were then treated with indicated inhibitors or agonists in each experiment for 20 h. Cells were washed twice with Agilent Seahorse XF Media (Agilent) supplemented with 1 mM pyruvate, 2 mM L-glutamine, and 10 mM D-glucose; a final volume of 525 μl was placed in each well. Cells were then incubated in a 0% CO2 chamber at 37° C. for 1 h before being placed into a Seahorse XFe24 Analyzer (Agilent). For OCR and ECAR mitostress test experiments, cells were treated with 1 μM oligomycin, 2 μM carbonyl cyanide p-trifluoromethoxyphenylhydrazone (FCCP), and 0.5 μM rotenone/antimycin (indicated by three black arrows in each seahorse trace). For mitoFlex Fuel test experiments, cells were treated with UK5099 (200 μM) and BPTES (200 μM)/Etomoxir (80 μM). A total of three OCR and pH measurements were taken after each compound was administered. All Seahorse experiments were repeated at least three times unless otherwise indicated. All OCR and ECAR data were normalized to cell number per well using CyQUANT (ThermoFisher Scientific, Waltham, Mass., USA).

### Flow Cytometry

Human MDMs were plated in 10-cm plates at 10×10⁶ cells per well collected using 0.25% trypsin-EDTA at 37° C. Cells were washed with flow cytometry buffer (PBS with 2% FCS, 2 mM EDTA, and 25 mM HEPES, pH7.4), and then incubated with blocking buffer (5% mouse serum in flow cytometry buffer) for 15 min at 4° C. Cells were then stained with the desired antibodies for 30 min at 4° C. Dead cells were identified and excluded using 0.5 μg/ml propidium iodide. The following controls were used: unstained cells; single-stained cells; and dead cells. The cells were gated using forward and side scatter, as well as live/dead staining using 4,6-diamidino-2-phenylindole (Thermo Fisher Scientific). Cells were analyzed on a BD FACSAria II (BD Biosciences). Raw FCS files were analyzed with the FlowJo software.

TABLE 1
Flow Cytometry Antibodies

| | Species | | | | | |
|---|---|---|---|---|---|---|
| Marker | Reactivity | Channel | Catalog # | Dilution | Clone | Source |
| CD14 | Human | APC-Cy7 | 557831 | 1:50 | MφP9 | BD |
| | | | | | | Biosciences |
| CD64 | Human | FITC | 560970 | 1:50 | 10.1 | BD |
| | | | | | | Biosciences |
| CD206 | Human | PE | 566281 | 1:50 | 19.2 | BD |
| | | | | | | Biosciences |
| CD68 | Human | PE-Cy7 | 565595 | 1:200 | Y1/82A | BD |
| | | | | | | Biosceinces |
| CD163 | Human | 563889 | 555660 | 1:50 | GHI/61 | BD |
| | | | | | | Biosciences |
| CD86 | Human | APC | 555660 | 1:50 | FUN-1 | BD |
| | | | | | | Biosciences |
| CD80 | Mouse | BV421 | 564160 | 1:50 | 16-10A1 | BD |
| | | | | | | Biosciences |
| CD14 | Mouse | PE-Cy7 | 553740 | 1:50 | rmC5-3 | BD |
| | | | | | | Biosciences |
| EGR-2 | Mouse | APC | 1706691-82 | 1:50 | ERONGR2 | Invitrogen |
| CD71 | Mouse | FITC | 561936 | 1:50 | C2F2 | BD |
| | | | | | | Biosciences |
| CD86 | Mouse | BV605 | 563055 | 1:50 | GL1 | BD |
| | | | | | | Biosciences |

### Chemokine and Cytokine Multiplex Assay

Hippocampal lysates or mouse plasma were stored at −80° C. and cytokine analysis was carried out at the Human Immune Monitoring Core (Stanford University) or Eve Technologies (Calgary, Alberta, Canada) using Luminex mouse 39-plex kits and Human 71-plex kits. Plates were read using a Luminex LabMap200 instrument or a MSD Chemiluminescence instrument with a lower bound of 100 beads per sample per measured cytokine. Each sample was tested in triplicate. Mean fluorescence intensity (MFI) was averaged over duplicate wells for each cytokine per sample on each plate. All were transcardially perfused prior to isolation of hippocampi and other brain tissue.

### Novel Object Location (Object Location Memory Task)

The Novel Object Location protocol was adopted from Wimmer et al. with minor modifications. Mice interacted with the chamber (a 16 in×16 in×15 in white box made from PVC) over the course of 2 days involving 1 habituation period, 3 training sessions, and 1 testing session. On the day of training (day 1) mice were placed in the middle of an empty chamber and given 5 minutes to explore the chamber. Mice were then placed in an independent holding cage for an inter-training interval (ITI) of 3 minutes. The objects used were a plastic bottle and seasoning shaker of similar size (3 in H×1 in W×1 in L). After the habituation session, the mice then underwent three 10-minute training sessions each with a 3 min ITI in between sessions. 24 h after the last training session, a testing session was conducted in which one of the objects was displaced to a new location. Animals were recorded a JVC Everio HD camcorder GZE200 and analyzed with Kinovea video tracking software. Exploration of the objects was defined as the amount of time mice were oriented toward an object with its nose within 1 cm of it, and was scored by an experimenter blind to experimental group.

### Barnes Maze

The Barnes maze protocol was adopted from Attar, A. et al. PLOS ONE 8, e80355 (2013) incorporated herein by reference) with minor modifications. The maze was made from a circular, 8-mm thick, white PVC slab with a diameter of 36 inches. Twenty holes with a diameter of 3 inches were made on the perimeter at a distance of 1 inch from the edge. This circular platform was then mounted on top of a rotating stool, 30 inches above the ground.

The escape cage was made by using a mouse cage and assembling a platform and ramp 2 inches below the surface of the maze. The outside of the walls of the cage were covered with black tape so as to prevent light for entering the escape cage. The maze placed in the center of a dedicated room and two 120 W lights were placed on the edges of the room facing towards the maze to provide an aversive stimulus for the mice. Eight simple colored-paper shapes (squares, rectangles, and circles) were mounted on the walls of the room as visual cues.

After testing each mouse, the maze was cleaned with 70% ethanol and rotated clockwise after every mouse to avoid intra-maze odor or visual cues. All sessions were recorded using a JVC Everio HD camcorder GZ-E200 and analyzed with Kinovea video tracking software.

The animals interacted with the Barnes maze in three phases: habituation (day 1), training (days 2-3), and probe (day 4). Before starting each experiment, mice were acclimated to the testing room for 1 h. Then all mice from one cage (n=4-5) were placed in individual holding cages where they remained until the end of their testing sessions each day. On habituation day, the mice were placed in the center of the maze within a vertically oriented black PVC pipe 4 inches in diameter and 7 inches in height for 15 seconds. The mice were then guided slowly to the hole that lead to the escape cage over the course of 10-15 seconds. The mice were given 3 minutes to independently enter the target hole, and if they did not, they were nudged with the PVC pipe to enter. The 120 W lights were then shut off and mice were allowed to rest in the escape cage for 2 minutes.

The training phase occurred 24 h after the habituation phase and was split across 2 days (days 2 and 3), with 3 trials on the first day and 2 trials on the second day. During each trial, the mice were placed in the center of the maze within the PVC pipe for 15 seconds and after allowed 3 minutes to explore the maze. If mice found and entered the target hole before 3 minutes passed, the lights were shut off and the training trial ended. Mice were allowed to rest in the escape cage for 2 minutes. If at the end of the three minutes the mice had not entered the target hole, they were nudged with the PVC pipe. A total of 5 trials were conducted. During each trial, latency (time) to enter the target hole as well as distance traveled were recorded.

The probe phase occurred 24 h after the training phase and was conducted on the last day (day 4). Mice were placed in the center of the maze within the PVC pipe for 15 seconds and after allowed 3 minutes to explore the maze. The probe session ended whenever the mouse entered the target hole or if 3 minutes had passed. During the probe phase, measures of time spent per quadrant, latency to enter the target hole, and distance traveled were recorded.

### Electrophysiology

To measure the cellular mechanism of learning and memory, a modified protocol previously described (Latif-Hernandez et al., Frontiers in cellular neuroscience 10, 252, (2016), incorporated herein by reference) was used. Mice were euthanized by cervical dislocation, and hippocampus (HC) was rapidly dissected out into ice-cold (4° C.) artificial cerebrospinal fluid (ACSF), saturated with carbogen (95% O2/5% CO2). ACSF consisted of (in mM): 124 NaCl, 4.9 KCl, 24.6 NaHCO₃, 1.20 KH₂PO₄, 2.0 CaCl₂, 2.0 MgSO₄, 10.0 glucose, pH 7.4. Transverse hippocampal slices (350 μm thick) were prepared from the dorsal area of the HC with the McIlwain tissue chopper and transferred to a recovery chamber for at least 1.5 hours with oxygenated ACSF at room temperature before being placed into a submerged-type chamber where they were kept at 32° C. and continuously perfused with ACSF at a flow-rate of 1.5 ml/min. Slices were carefully positioned on a R6501A multi-electrode array (Alpha MED Scientific Inc., Osaka, Japan) with electrodes arrayed in an 8×2 matrix with interpolar distance of 150 μm; each matrix measured 50 μm×50 μm. After 30 min incubation, the field excitatory postsynaptic potentials (fEPSPs) in CA1 were recorded by stimulating downstream electrodes in the CA1 and CA3 regions along the Schaffer collateral pathway. Signals were acquired using the MED64 System (AlphaMED Sciences, Panasonic). The time course of the fEPSP was calculated as the descending slope function for all experiments. Input/output (I/O) curves were established by applying increasing stimulus currents to the pathway from 10 μA to 90 μA (in 5 μA increments) and recording evoked responses. After I/O curves had been established, the stimulation strength was adjusted to elicit a fEPSP slope at 35% maximal value, which was maintained throughout the experiment. During baseline recording, a single response was evoked at a 30 seconds interval for at least 20 minutes. To induce a strong form of LTP, three episodes of theta-burst-stimulation (TBS) were employed, each TBS consisting of 10 burst of four stimuli at 100 Hz separated by 200 ms (double pulse width) followed by recording evoked responses 1 minute post-LTP induction and continued every 30 seconds until the end of the experiments. Experiments of control and transgenic mice were interleaved with each other. The mean baseline fEPSP value was calculated and percentage change from baseline after the TBS was analyzed for LTP.

### Quantitative Immunoblotting

Quantitative immunoblotting was carried out as described (Johansson, J. U. et al. The J. Clin. Investig. 125, 350-364, (2015), incorporated herein by reference). Mouse anti-β-actin (1:10,000; Sigma-Aldrich) was used as an internal loading control. Densitometry quantification was carried out using ImageJ (NIH). Antibodies and their concentrations are listed:

TABLE 2
Western Blot Antibodies

| Antibody | Source | Catalog No. | Dilution |
|---|---|---|---|
| Anti-Synapsin | Millipore Sigma | AB1543P | 1:1000 |
| Anti-SNAP25 | Abcam | ab41455 | 1 μg/ml |
| Anti-P5D95 | Abcam | ab2723 | 1 μg/ml |
| Anti-CamKIIa (pan) | Cell Signaling | 3362S | 1:1000 |
| | Technology | | |
| Anti-EP1 | Cayman Chemical | 101740 | 1:200 |
| Anti-EP2 | Abcam | Ab167171 | 1:1000 |
| Anti-EP3 | Cayman Chemical | 101760 | 1:200 |
| Anti-EP4 | Santa Cruz | Se-55596 | 1:100 |
| Anti-p(Ser473)AKT | Cell Signaling | 4060S | 1:1000 |
| | Technology | | |
| Anti-AKT (pan) | Cell Signaling | 2920S | 1:1000 |
| | Technology | | |
| Anti-p(Ser9)GSK3β | Cell Signaling | 9336 | 1:500 |
| | Technology | | |
| Anti-GSK3β | Abcam | ab93926 | 1:500 |
| Anti- | Millipore Sigma | 07-817 | 1:500 |
| p(Ser641,645,649)GYS1 | | | |
| Anti-GYS1 [Human] | ThermoFisher | MA5-15802 | 1:500 |
| | Scientific | | |
| Anti-GYS1 [Mouse] | ThermoFisher | MA5-15022 | 1:1000 |
| | Scientific | | |
| Anti-TOM20 | Santa Cruz | sc-17764 | 1:500 |
| Anti-VDAC | Abcam | ab14734 | 1:500 |
| Anti-TIM17 | Santa Cruz | sc-271152 | 1:500 |
| Anti-OPA1 | BD Biosciences | 612607 | 1:1000 |
| Anti-MFN2 | Abnova | 11325-6A8 | 1:1000 |
| Anti-p(Ser616)DRP1 | Cell Signaling | 4494S | 1:250 |
| | Technology | | |
| Anti-FIS1 | Proteintech | 10956-1-AP | 1:1000 |
| Anti-COX2 | Cayman Chemical | 160126 | 1:1000 |
| Anti-PGES | Abcam | Ab62050 | 1:500 |
| Anti-β-Tubulin | EMD Millipore | 05-661 | 1:5000 |
| Anti-β-Actin | Millipore Sigma | A5441 | 1:10000 |

### Glycogen Quantification

Glycogen was quantified using a commercially available colorimetric kit, Cat. No. ab65620, Abcam.

### Peritoneal Macrophages

Peritoneal macrophages were collected from 2-4, 6, and 22-24 month-old) Cd11bCre;EP2^{lox/lox},

Cd11bCre, and WT mice. Mice were injected intraperitoneally with 1.5 ml 3% (w/v) thioglycolate medium (BD Biosciences), and primary macrophages were isolated 3-4 days later by flushing with ice-cold 1×PBS buffer (Corning). Cells were seeded at a density of 3×10⁶ cells per well in DMEM supplemented with 10% heat-inactivated fetal bovine serum (FBS; Sigma-Aldrich), 100 U ml⁻¹ penicillin and streptomycin, and maintained at 5% CO₂ at 37° C. After overnight culture, cells were washed twice with medium to remove nonadherent cells.

### Neuron & Astrocyte Culture

Hippocampi were dissected from embryonic Day 17.5 mice embryos, dissociated using trypsin (2 mg/ml) and DNase I (0.6 mg/ml), and plated at a density of 100, 000 cells per well in Seahorse XF24 culture plate coated with poly-L-lysine. Neurons were maintained in Neurobasal® medium, B27 (Invitrogen), and penicillin—streptomycin (Invitrogen) at 37° C. in a humidified atmosphere containing 5% CO₂. Media was refreshed twice weekly by replacing half the media with fresh media. After 12-14 days in vitro, cells underwent real-time oxygen consumption analysis with the Seahorse XFe24 machine and the MitoStress test kit.

Primary astrocyte cultures were prepared from cerebral cortices of postnatal day 1-2 C57BL/6J mice. In brief, dissociated cortical cells were suspended in DMEM/F12 50/50 (Life Technologies, cat. no. 11320-033) containing 25 mM glucose, 4 mM glutamine, 1 mM sodium pyruvate and 10% FBS, and plated on uncoated 75-cm² flasks at a density of 1.5×10⁵ cells/cm². Monolayers of astrocytes were obtained 12-14 days after plating. Cultures were gently shaken, and floating cells (microglia) were collected, resulting in more than 95% pure culture of astrocytes. Astrocytes were dissociated by trypsinization and then reseeded at 4×10⁴ cells per well in a XF24-well cell culture microplate and cells underwent real-time oxygen consumption analysis with the Seahorse XFe24 machine and the MitoStress test kit.

### Human Monocyte-Derived Macrophages

Peripheral blood mononuclear cells from de-identified healthy donors (young<35 years old, aged>65 years old) were obtained from the Stanford Blood Center and transferred to 50 ml conical tubes. Samples were diluted with 20 ml PBS and layered onto 10 ml of Ficoll-Paque (GE Healthcare) using a Pasteur pipette. Tubes were centrifuged at 1,500 r.p.m. for 25 min without brake at 20° C. The mononuclear cell layer was transferred to a new 50 ml conical tube, resuspended in 50 ml 1×PBS and centrifuged at 1500 r.p.m. for 10 min, repeated twice. After centrifugation, monocytes were isolated using a Monocyte Isolation Kit, human (MACS; Miltenyi Biotech). Cells were then plated 10×10⁶ per 10-cm petri dish and differentiated for 7 days in Roswell Park Memorial Institute (RPMI) media supplemented with 10% FBS, 1% penicillin-streptomycin, and 50 ng ml⁻¹ M-CSF (Peprotech).

### LC/MS Measurement of Metabolites

Isotope labeling was performed as previously described (Su et al. Anal. Chem. (2017), incorporated herein by reference). Labeled compounds U-₁₃C-Glucose (Cambridge Isotope Laboratories) were added to customized RPMI media lacking Glucose (customized RPMI 1640 Medium+Gibco GlutaMAX supplement+HEPES; Thermo Fisher Scientific) or prepared in 0.9% saline for oral gavage.

Water Soluble Metabolites: Human MDMs were grown on 10-cm plates (Corning). For steady-state labeling of metabolites, U-¹³C-Glucose (11.1 mM) labeled medium was replaced every day and then 20 h before metabolic analysis at which point cellular metabolism was quenched by rapidly cooling cells on dry ice. Cells were washed with 1×PBS twice by aspirating media and immediately adding 1 ml-80° C. 80:20 methanol/water. After 20 min of incubation on dry ice, the resulting mixture was scraped, collected into a centrifuge tube, and centrifuged at 10,000 g for 5 min at 4° C. Pellets were then extracted again with 500 μl-80° C. 80:20 methanol/water and incubated for 5 min, centrifuged at 10,000 g for 5 min at 4° C. Both extractions were combined into a 1.5 ml microcentrifuge tube. The supernatants were centrifuged at 16,000 g for 20 minutes to remove any residual debris before analysis. Supernatants were analyzed within 24 hours by liquid chromatography coupled to a mass spectrometer (LC-MS). The LC-MS method involved hydrophilic interaction chromatography (HILIC) coupled to the Q Exactive PLUS mass spectrometer (Thermo Scientific). The LC separation was performed on a XBridge BEH Amide column (150 mm 3 2.1 mm, 2.5 mm particle size, Waters, Milford, Mass.). Solvent A is 95%: 5% H₂O: acetonitrile with 20 mM ammonium bicarbonate, and solvent B is acetonitrile. The gradient was 0 min, 85% B; 2 min, 85% B; 3 min, 80% B; 5 min, 80% B; 6 min, 75% B; 7 min, 75% B; 8 min, 70% B; 9 min, 70% B; 10 min, 50% B; 12 min, 50% B; 13 min, 25% B; 16 min, 25% B; 18 min, 0% B; 23 min, 0% B; 24 min, 85% B; 30 min, 85% B. Other LC parameters are: flow rate 150 ml/min, column temperature 25° C., injection volume 10 mL and autosampler temperature was 5° C. The mass spectrometer was operated in both negative and positive ion mode for the detection of metabolites. Other MS parameters are: resolution of 140,000 at m/z 200, automatic gain control (AGC) target at 3e6, maximum injection time of 30 ms and scan range of m/z 75-1000. Data were analyzed via the MAVEN software, and isotope labeling was corrected for natural ¹³C abundance in the tracer experiments. For identification and isolation of hexose phosphates and glycolytic intermediates, capillary electrophoresis mass spectroscopy was used as described (Yamashita et al. PLOS ONE 9, e86426 (2014), incorporated herein by reference). In brief, adherent cells on dishes were washed with 5% mannitol aqueous solution at room temperature. The cells were immersed in 400 μL of methanol for 30 seconds, and 275 μL of the Internal Standard Solution (10 μM, Solution ID: H3304-1002, Human Metabolome Technologies) for 30 seconds. The extraction liquid was centrifuged at 2,300×g for 5 minutes at 4° C. The supernatant (400 μL) was centrifugally filtered at 9,100×g for 4 hours at 4° C. through a 5-kDa cutoff filter (Millipore) to remove proteins, and then the filtrate was lyophilized and suspended in 25 μL of Milli-Q water. The metabolite suspension was analyzed by CE-TOFMS using an Agilent capillary electrophoresis (CE) system equipped with an Agilent 6210 TOFMS, an 1100 isocratic high-performance liquid chromatography pump, a G1603A CE-MS adapter kit and a G1607A CE-electrospray ionization-mass spectrometry (ESI-MS) sprayer kit (Agilent Technologies, Waldbronn, Germany). The system was controlled using G2201AA ChemStation software version B.03.01 for CE (Agilent).

### Nucleofection

Plasmid-containing human GYS1 shRNA (Table 3) or Scr (2 μg) (HuSH shRNA shGYS1 Lenti Cloning Vector [pGFP-C-shLenti]) were incubated with a mixture of nucleofection solution and P4 primary cell supplement (82 μl:18 μl nucleofection solution:supplement) and placed in nucleofection cuvettes. A total of 1×10⁶ human MDMs were added to each cuvette and subjected to program Y-010 for the Nucleofector 2b Device (Lonza). Immediately afterwards, 500 μl of DMEM (preincubated at 37° C. under 5% CO₂ and supplemented with 20% FBS and 1% penicillin-streptomycin) was added. Cells were then plated in 10-cm plates and incubated 37° C. under 5% CO₂ for 8 h before GYS1 protein expression was analyzed by quantitative immunoblotting.

TABLE 3
Human GYS1 shRNA sequences

| shRNA | Vector | ShRNA sequence |
|---|---|---|
| shRNA #1 | pGFP-C-shLenti | 5′-UCAACAGCAGUGCCGACCGA |
| | | CCGACCGUGAAGGUG-3′ |
| shRNA #2 | pGFP-C-shLenti | 5′-GAUCGAAAGACAGCCUGGUC |
| | | A-3′ |

### Phagocytosis Assay

Human MDMs were grown in 10-cm plates at 10×10⁶ cells per well and then trypsinized using 0.25% trypsin-EDTA at 37° C. Cells were then plated in 96-well plates at 80,000 cells per well and the Vybrant Phagocytosis Assay Kit (Thermo Fisher Scientific) with E. coli particles was carried out following the manufacturer's protocol.

### PGE₂ LC/MS Detection

Brain tissue was homogenized with 500 μl of MeOH:formic acid (100:0.2) containing internal standard consisting of a mixture of deuterium-labeled Prostaglandins, using microtip sonication. The samples were submitted to solid phase extraction using an Oasis HLB cartridge (5 mg; Waters, Milford, Mass.) (Yamada et al. J. Chromatog. B 995-996, 74-84 (2015); Kita et al., Analyt. Biochem. 342, 134-143 (2005), incorporated herein by reference). Briefly, samples were diluted with water:formic acid (100:0.03) to give a final MeOH concentration of _{˜}20% by volume, applied to preconditioned cartridges, and washed serially with water:formic acid (100:0.03), water:ethanol:formic acid (90:10:0.03), and petroleum ether. Samples were then eluted with 200 μl of MeOH:formic acid (100:0.2). The filtrate was concentrated with a vacuum concentrator (SpeedVac, Thermo). The concentrated filtrate was dissolved in 20 μL of methanol and used for LC-MS/MS. The amount of PGs in brain tissue was quantified using the method of Yamada et al. Briefly, a triple-quadrupole mass spectrometer equipped with an electrospray ionization (ESI) ion source (LCMS-8060; Shimadzu Corporation, Kyoto, Kyoto, Japan) was used in the positive and negative-ESI and multiple reaction monitoring (MRM) modes.

### PGE₂ ELISA Detection

Cell lysates and medium were prepared according to manufacturer's instructions for PGE2 detection by ELISA (Catalog No. KGE004B, Research & Diagnostic Systems Inc., Minneapolis, Minn., USA).

### Rapid Microglial Isolation

Mice were transcardially perfused with ice cold PBS containing EDTA. Brains were minced with a razor blade and a single cell suspension obtained by dounce homogenization in a solution of HBSS containing HEPES, glucose, and DNAse I. The suspension was filtered through a 70 um strainer. Myelin was removed using myelin removal beads (Miltenyi Biotec, Bergisch Gladbach, Germany). Cells were stained with CD45-FITC (BioLegend 30-F11) and CD11b-PE Dazzle 594 (BioLegend M1/70) for microglia identification through flow cytometry. Live cells were identified by 7-AAD exclusion.

### Synaptic Mitochondria Isolation

Synaptic mitochondria were isolated as described (Gauba et al., Neurobiol. Dis. 121, 138-147 (2019), incorporated herein by reference). Briefly, brain cortices were removed and added to cold freshly prepared 9 ml of mitochondrial isolation buffer (225 mM mannitol, 75 mM sucrose, 2 mM K2PO4, 0.1% BSA, 5 mM HEPES, 1 mM EGTA (pH 7.2). The tissue were homogenized using a dounce homogenizer. The resultant homogenate was centrifuged at 13,00 g at 4° C. and layered on top of 3×2-ml discontinuous gradient of 15%, 23% and 40% Percoll (GE) and centrifuged at 34,000 g for 14 minutes. Following centrifugation, the band between 15% and 23% containing synaptosomes and band between 23% and 40% containing nonsynaptic mitochondria were removed and subjected to wash in IB with 0.02% digitonin. The isolates were then pelleted by centrifugation at 16,500 g for 15 minutes. The pellets were resuspended in IB and layered over another discontinuous gradient similar to described above. Band between 23% and 40% containing synaptic mitochondria was obtained and washed in ice cold IB. Protein estimation was performed using BioRad Bradford assay (bioRad Laboratories). Isolated mitochondria were immediately used for analysis. 10 ug of freshly isolated synaptic mitochondria were plated in XFe24 cell culture microplates in a volume of 50 μl mitochondrial assay solution (MAS-70 mM Sucrose, 220 mM mannitol, 10 mM KH₂PO₄, 5 mM MgCl₂, 2 mM HEPES, 1 mM EGTA and 0.2% BSA with 10 mM succinate and 2 uM rotenone) and attached to the wells by spinning down the plates at 2000 rpm at 4° C. After attaching mitochondria to the plate wells, volume of wells were made up to 450 uL of MAS containing substrate. In the meantime, Seahorse XF24 Flux Analyzer was equilibrated to 37° C. overnight a day before the assay. The final concentrations of substrates and inhibitors added to the wells were 4 mM ADP, 2.5 ug/ml Oligomycin A, 4 uM FCCP and 4 uM Antimycin A. The coupling assay were run in 2-3 replicate wells for each independent biological sample. XFe24 data were collected according to Seahorse software. The point to point run for each wells were exported and statistical analysis was conducted in PRISM (GraphPad Software) using two tailed unpaired t-tests.

### Measurement of PF04418948 in Plasma and Brain

Presence of PF04418948 was measured in aged (20-22 mo) mice treated with PF04418948 2.5 mg/kg/d for 6 weeks in plasma and perfused brain by LC/MS/MS at Quintara Discovery (Hayward, Calif., USA). In brief, brain samples were first homogenized in 2 volumes of ice-cold water, then further 2 fold diluted in blank plasma. An aliquot of 20 μL of each plasma sample or plasma diluted tissue homogenate was treated with 100 μL of acetonitrile containing internal standard (Terfenadine). The mixture was vortexed on a shaker for 15 minutes and subsequently centrifuged at 4000 rpm for 15 minutes. An aliquot of 70 μL of the extract was transferred to an injection plate and reconstituted in 70 μL of water for LC/MS/MS injection. Calibration standards and quality control samples were prepared by spiking the testing compound into blank plasma followed by processing with the samples.

### Nanostring Transcriptomics

For gene expression analysis on the NanoString nCounter system, 100 ng of RNA was hybridized to a multiplexed nucleotide probe pool for 16 hours at 65° C. Enriched targets were purified and quantified using the nCounter MAX Analysis System. NanoString data analysis was performed by Canopy Biosciences (St. Louis, Mo.). Raw counts were normalized using the geometric mean of both positive control probes (technical variability) and housekeeping gene probes (assay input variability). Normalized data was uploaded to the interactive analysis platform Rosalind (https://rosalind.onramp.bio/), with a HyperScale architecture developed by OnRamp BioInformatics, Inc. (San Diego, Calif.) Read Distribution percentages, violin plots, identity heatmaps, and sample MDS plots were generated as part of the QC step. The limma R library1 was used to calculate fold changes and p-values and perform optional covariate correction. Clustering of genes for the final heatmap of differentially expressed genes was done using the PAM (Partitioning Around Medoids) method using the fpc R library2 that takes into consideration the direction and type of all signals on a pathway, the position, role and type of every gene, etc. Functional enrichment analysis of pathways, gene ontology, domain structure and other ontologies was performed using HOMER3. Several database sources were referenced for enrichment analysis, including Interpro4, NCBI5, MSigDB6,7, REACTOME8, WikiPathways9. Enrichment was calculated relative to a set of background genes relevant for the experiment.

### Statistical Analyses

Data are expressed as the mean±s.e.m., unless otherwise indicated. Statistical comparisons were made in the Prism software using a Student's t-test (for two groups meeting the normal distribution criteria, according to the Shapiro-Wilk normality test), Mann-Whitney U-test (for two groups not meeting the normal distribution criteria), or analysis of variance (ANOVA) with Tukey's multiple comparison test (for groups across variables, with multiple comparisons between groups). Data were subjected to Grubbs' test to identify the presence or absence of outlier data points. For all tests, P<0.05 was considered significant, except for targeted metabolomics in which Q<0.05.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

One aspect of the disclosure, therefore, encompasses embodiments of a method for reducing inflammation in a subject, wherein the inflammation is associated with neurological or cognitive decline in the subject, comprising inhibiting an EP2 (Prostaglandin E2 receptor 2)-generated signal in the subject by contacting EP2 with an EP2 antagonist.

In some embodiments of this aspect of the disclosure, inhibiting the EP2 signal comprises administering to the subject a composition comprising a brain-penetrant EP2 antagonist, a peripheral EP2 antagonist, or both.

In some embodiments of this aspect of the disclosure, the EP2 antagonist is a small molecule antagonist.

In some embodiments of this aspect of the disclosure, the EP2 is in aged human monocyte-derived macrophages.

In some embodiments of this aspect of the disclosure, the brain-penetrant EP2 antagonist is compound 52.

In some embodiments of this aspect of the disclosure, the peripheral EP2 antagonist is PF04418948.

Another aspect of the disclosure encompasses embodiments of a method for reducing cognitive decline in a subject, comprising inhibiting an EP2-generated signal by administering a composition comprising an EP2 antagonist to a subject in need thereof.

In some embodiments of this aspect of the disclosure, the EP2-generated signal is a myeloid EP2-generated signal.

In some embodiments of this aspect of the disclosure, the myeloid EP2-generated signal is inhibited in aged mammalian monocyte-derived macrophages.

In some embodiments of this aspect of the disclosure, the composition is administered to the mammal peripherally.

In some embodiments of this aspect of the disclosure, administering is oral or intravenous.

Yet another aspect of the disclosure encompasses embodiments of a pharmaceutical composition comprising an EP2 antagonist and a pharmaceutically acceptable carrier, wherein the pharmaceutical composition is formulated to deliver an effective dose of the antagonist to the mammal that inhibits an EP2-generated signal in the cells thereof.

In some embodiments of this aspect of the disclosure, the therapeutically effective amount is effective to reduce brain and/or peripheral myeloid EP2-generated signaling.

What is claimed is:

1. A method for reducing inflammation in a subject, wherein the inflammation is associated with neurological or cognitive decline in the subject, comprising inhibiting an EP2 (Prostaglandin E₂ receptor 2)-generated signal in the subject by contacting EP2 with an EP2 antagonist.

2. The method of claim 1, wherein inhibiting the EP2 signal comprises administering to the subject a composition comprising a brain-penetrant EP2 antagonist, a peripheral EP2 antagonist, or both.

3. The method of claim 1, wherein the EP2 antagonist is a small molecule antagonist.

4. The method of claim 1, wherein the EP2 is in aged human monocyte-derived macrophages.

5. The method of claim 4, wherein the brain-penetrant EP2 antagonist is compound 52.

6. The method of claim 4, wherein the peripheral EP2 antagonist is PF04418948.

7. A method for reducing cognitive decline in a subject, comprising inhibiting an EP2-generated signal by administering a composition comprising an EP2 antagonist to a subject in need thereof.

8. The method of claim 7, wherein the EP2-generated signal is a myeloid EP2-generated signal.

9. The method of claim 8, wherein the myeloid EP2-generated signal is inhibited in aged mammalian monocyte-derived macrophages.

10. The method of claim 7, wherein the composition is administered to the mammal peripherally.

11. The method of claim 7, wherein the administering is oral or intravenous.

12. A pharmaceutical composition comprising an EP2 antagonist and a pharmaceutically acceptable carrier, wherein the pharmaceutical composition is formulated to deliver an effective dose of the antagonist to the mammal that inhibits an EP2-generated signal in the cells thereof.

13. The pharmaceutical composition of claim 12, wherein the therapeutically effective amount is effective to reduce brain and/or peripheral myeloid EP2-generated signaling.


SEQUENCE LISTING (2 sequences)

SEQ ID NO: 1 - RNA, 35 nt, Artificial sequence
  shRNA #1 pGFP-C-shLenti
ucaacagcagugccgaccgaccgaccgugaaggug

SEQ ID NO: 2 - RNA, 21 nt, Artificial sequence
  shRNA #2 pGFP-C-shLenti
gaucgaaagacagccugguca